(19) United States
(12) Patent Application Publication
(10) Pub. No.: US 2010/0069481 A1
(43) Pub. Date: Mar. 18, 2010

(54) METHODS AND COMPOSITIONS FOR THE TREATMENT OF CANCER

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: Bionovo, Inc., Emeryville, CA (US)

(21) Appl. No.: 12/553,892

(22) Filed: Sep. 3, 2009

Related U.S. Application Data
(60) Provisional application No. 61/094,012, filed on Sep. 3, 2008.
(60) Provisional application No. 61/162,988, filed on Mar. 24, 2009.
(60) Provisional application No. 61/172,639, filed on Apr. 24, 2009.

(51) Int. Cl. A61K 31/352; A61P 35/00; A61P 35/04

(52) U.S. Cl. 514/456

(57) ABSTRACT

Isolated compounds and combinations of isolated compounds isolated from Scutellaria barbata D. Don are effective in the generation of reactive oxygen species, induction of DNA damage and induction of apoptosis in cancer cells. The compounds and combinations may be prepared as pharmaceutical compositions for administration to mammals, such as humans, for the treatment of solid cancers, such as epithelial cancers. Such epithelial cancers include breast cancer and ovarian cancers.

### CROSS-REFERENCE

This application claims the benefit of U.S. Provisional Application Nos. 61/094,012, filed, Sep. 3, 2008, 61/162,988, filed Mar. 24, 2009; and 61/172,639, filed Apr. 24, 2009, each of which is incorporated herein by reference in its entirety.

### BACKGROUND OF THE INVENTION

While advances in early detection and adjuvant therapy for breast cancer have had a favorable impact on patient survival in general, patients who develop advanced metastatic breast cancer are generally likely to face a less favorable prognosis. Commonly used hormonal and chemotherapeutic agents can lead to transient regression of tumors and can also palliate symptoms related to cancer. However, these treatments are often accompanied by toxicities and intolerable side effects and eventually become ineffective in controlling advanced stage breast cancer and its symptoms. Improvements in breast cancer survival are modest, even with newer targeted biological agents. Moreover, in most metastatic cancers, resistance to available conventional treatment ultimately develops, or patients experience excessive side effects.

It is interesting to note that greater than 60% of all chemotherapeutic agents used in the treatment of breast cancer are derived from natural substances (Newman 2003). A fairly recent example is the development of taxanes from the Pacific yew tree, Taxus brevifolia. Throughout the world, it is estimated that approximately 80% of the world population still relies on botanical medicine as the primary source of therapy. In the West, botanical medicine is considered a popular form of complementary and alternative medicine among patients diagnosed with cancer. However, few clinical trials have been conducted to firmly assess the safety and efficacy of botanical agents for the treatment of breast cancer, despite anecdotal case reports of cures and clinical efficacy in women who have relied solely on botanical medicine for treatment. It has previously been shown that the aqueous extract of Scutellaria barbata can lead to growth inhibition of breast cancer cell lines in vitro (“Antiproliferative activity of Chinese medicinal herbs on breast cancer cells in vitro,” Anticancer Res., 22(6C):3843-52 (2002)). BZL101, a concentrated aqueous extract of Scutellaria Barbata, was evaluated for antiproliferative activity on five breast cancer cell lines (SK-BR-3, MCF7, MDA-MB-231, BT-474, and MCNeuA). These cell lines represent important prognostic phenotypes of breast cancer expressing a range of estrogen and HER2 receptors. BZL101, tested at a 1:10 dilution (15 μg/ml), demonstrated >50% growth inhibition on four of the five cell lines (Campbell, 2002). BZL101 showed >50% growth inhibition on a panel of lung, prostate and pancreatic cancer cell lines. BZL101 at the same dose did not cause >25% of growth inhibition on normal human mammary cells (HuMEC), demonstrating selectivity to cancer cells (Table 1). More so, BZL101 had a mild mitogenic effect on normal human lymphocytes. In cell cycle analysis, BZL101 caused an S phase burst and G1 arrest. BZL101 also attenuated mitochondrial membrane potential causing caspase-independent high molecular grade (HMG) apoptosis.

There is a need for therapies for treatment of patients having metastatic cancers. There is also a need for therapies with reduced, and more specifically minimal, toxicity for patients having metastatic cancers. In particular, there is a need for novel therapies with relatively low toxicity for the treatment of metastatic solid tumors, such as epithelial tumors, and more particularly breast and ovarian cancers.

These and other needs are met by embodiments of the invention.

### SUMMARY OF THE INVENTION

The inventor has found that an extract of Scutellaria barbata D. Don is well-tolerated at doses much higher than previously reported. The extract of Scutellaria barbata D. Don is well-tolerated at dosages of at least about 20 g of soluble material extracted from Scutellaria barbata D. Don. Furthermore, the inventor has found that the extract of Scutellaria barbata D. Don may be conveniently provided in a dosage unit suitable for administration to a patient. Thus, in some embodiments, there is provided a dosage unit comprising at least about 20 g of soluble matter extracted from Scutellaria barbata D. Don. In some embodiments, the unit dose further comprises at least one excipient, especially at least one excipient other than water, and in particular at least one taste-masking agent, sweetener or both. In particular embodiments, the dosage unit is in an form suitable for oral administration, e.g. an aqueous (water-based) composition or a dry powder suitable for reconstitution with water. The inventor has found that the dosage unit is suitable for administration to a cancer patient, especially a cancer patient suffering from breast cancer or a gynecological cancer, such as uterine cancer.

The inventor having determined that a dose of at least about 20 g per day of soluble matter extracted from Scutellaria barbata D. Don is well-tolerated and effective for the treatment of cancer, especially breast cancer. Thus, the invention provides a method of treating cancer, comprising administering to a cancer patient at least about 20 g per day of soluble matter extracted from Scutellaria barbata D. Don. In some embodiments, the cancer is selected from breast cancer and one or more gynecological cancers. In some embodiments, the method includes administering to the patient about 20 g per day to about 200 g per day of soluble matter extracted from Scutellaria barbata D. Don.

The inventor has also determined that addition of an excipient, such as a taste-masking agent, to a high dose of a pharmaceutical composition comprising an extract of Scutellaria barbata D. Don attenuates the bitter taste of the extract. As the inventor has found that high doses of Scutellaria barbata D. Don (e.g. at least about 20 g soluble matter per dose or per day) are well-tolerated, but relatively unpalatable, the inventor has found the addition of a taste-masking agent or other agent is desirable for making the composition palatable for consumption of high dosages of Scutellaria barbata D. Don extract, such as for the treatment of cancer. Thus, embodiments described herein provide a pharmaceutical composition (e.g. for the treatment of cancer, especially breast or gynecological cancer) comprising at least one excipient other than water (such as at least one taste-masking agent, sweetener or both), and one or more members of the group consisting of Luteolin, Apigenin, Scutellarein, and Scutellarin, which are the anti-cancer actives that the inventor has identified as being necessary for the anti-cancer activity of an extract of Scutellaria barbata D. Don. The inventor has also found that high molecular weight compounds, e.g. compounds with high molecular weights (e.g. molecular weights greater than 1,000-10,000 grams/mole) add to the bulk of the composition without conferring any substantial activity, and tend to cause stomach upset, gas, bloating and/or diarrhea. Thus, in some embodiments, the pharmaceutical composition is depleted of high molecular weight compounds, and in some embodiments is substantially free of high molecular weight compounds. In some embodiments, the composition contains a combination of Luteolin, Apigenin, Scutellarein, and Scutellarin, containing about 1 part Luteolin, about 1.1 parts Apigenin, About 4.8 parts Scutellarein and about 34 parts Scutellarin. (All “parts” determined by weight.) In some embodiments, the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin. In some embodiments, the composition contains about 1 g to about 200 g soluble matter, of which soluble matter about 1% to about 99% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin. In some embodiments, the composition is used to treat a breast cancers selected from one or more of is advanced breast cancer, metastatic breast cancer, treatment-refractory breast cancer, ER-negative breast cancer, PR-negative breast cancer, HER2-negative breast cancer, and/or triple-negative breast cancer.

Some embodiments described herein provide a method of treating cancer, especially one or more breast and/or gynecological cancers, comprising administering to the patient an effective amount of a composition comprising at least one excipient other than water (such as at least one taste-masking agent, sweetener or both), and one or more members of the group consisting of Luteolin, Apigenin, Scutellarein, and Scutellarin. In some embodiments, the composition is used to treat a breast cancers selected from one or more of is advanced breast cancer, metastatic breast cancer, treatment-refractory breast cancer, ER-negative breast cancer, PR-negative breast cancer, HER2-negative breast cancer, and/or triple-negative breast cancer. In some embodiments, the effective amount of the composition comprises at least 0.25 g of a combination of Luteolin, Apigenin, Scutellarein, and Scutellarin. In some embodiments, the effective amount of the composition comprises at least 0.27 g of a combination of Luteolin, Apigenin, Scutellarein, and Scutellarin. In some embodiments, the composition comprises at least about 0.35 g of the combination of Luteolin, Apigenin, Scutellarein, and Scutellarin. In some embodiments, the effective amount of the composition contains about 0.35 g-2 g, about 0.35 g-1.1 g, about 0.35-1 g, about 0.35 g to about 0.8 g of the combination of Luteolin, Apigenin, Scutellarein, and Scutellarin.

The inventor has found that a combination of Luteolin, Apigenin, Scutellarein, and Scutellarin is effective as a treatment for cancer, especially breast cancer. Thus, in some embodiments the invention provides a dosage unit comprising a combination of Luteolin, Apigenin, Scutellarein, and Scutellarin. In some embodiments, the dosage unit comprises at least about 0.25 g, at least about 0.27 g, or at least about 0.35 g of a combination of Luteolin, Apigenin, Scutellarein, and Scutellarin. In some embodiments, the dosage unit further comprises at least one excipient other than water, such as a taste masking agent, a sweetener or both. In some embodiments, the dosage unit is substantially free of high molecular weight compounds extracted from Scutellaria barbata D. Don. In some embodiments, the compositions are employed in a method of treating cancer, such as breast cancer and/or one or more gynecological cancers. In some embodiments, the cancer is a breast cancer, such as advanced breast cancer, metastatic breast cancer, treatment-refractory breast cancer, ER-negative breast cancer, PR-negative breast cancer, HER2-negative breast cancer, and/or triple-negative breast cancer.

The inventor has further discovered processes for making pharmaceutical compositions using the aerial portions of Scutellaria barbata D. Don as starting materials. Such processes are particularly useful for making compositions comprising Luteolin, Apigenin, Scutellarein, and Scutellarin. Thus, in some embodiments, the inventor has described a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; (d) optionally evaporating some, substantially all or all of the water from the refined extract or adding additional water to the refined extract; and (e) combining the refined extract with at least one pharmaceutically acceptable excipient other than water, to form the pharmaceutical composition. In some embodiments, the refined extract contains Apigenin, Luteolin, Scutellarein, and Scutellarin. In some embodiments, at least one pharmaceutically acceptable excipient other than water is selected from taste masking agents and sweeteners.

The inventor has found that removing at least some of the high molecular weight compounds extracted from Scutellaria barbata D. Don improves the clinical characteristics of the pharmaceutical composition. As a large amount of soluble matter extracted from Scutellaria barbata D. Don is inactive, reducing the amount of soluble matter by removing molecules having molecular weights above a predetermined cutoff will greatly reduce the bulk of a pharmaceutical composition derived from an extract of Scutellaria barbata D. Don. Additionally, a large part of the soluble matter extracted from Scutellaria barbata D. Don into water is soluble fiber, which is not absorbed in the intestines and tends to promote gastrointestinal upset, bloating, gas and diarrhea. Thus, removing at least part of the soluble fiber by reducing the burden of soluble matter extracted from Scutellaria barbata D. Don, while preserving the mixture of Luteolin, Apigenin, Scutellarein, and Scutellarin in the composition, results in an improved anti-cancer drug. Thus, in some embodiments, the invention provides a pharmaceutical composition comprising 1 part of a combination of Luteolin, Apigenin, Scutellarein, and Scutellarin and less than about 50 parts of high molecular weight compounds having molecular weights greater than a predetermined cutoff, wherein the predetermined cutoff is from 1,000 grams/mole to about 20,000 grams/mole.

The inventor has also discovered a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; (d) optionally evaporating some, substantially all or all of the water from the refined extract or adding additional water to the refined extract; and (e) combining the refined extract with a pharmaceutically acceptable excipient to form the pharmaceutical composition. 1 part Luteolin, about 0.61 to about 2 parts Apigenin, about 2.5 to about 9.4 parts Scutellarein, and about 15 to about 70 parts Scutellarin.

Some embodiments also provide a process of making a refined extract of Scutellaria barbata D. Don, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form the refined extract of Scutellaria barbata D. Don.

Some embodiments further provide a process of making a pharmaceutical composition, comprising combining at least one pharmaceutically acceptable excipient other than water with one or more members of the group consisting of Luteolin, Apigenin, Scutellarein, and Scutellarin to form the pharmaceutical composition. In some embodiments, at least one pharmaceutical excipient other than water is selected from taste masking agents and sweeteners.

Some embodiments provide a process of making a pharmaceutical dosage unit comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form a refined extract; and (d) combining the refined extract with at least one excipient other than water to form the pharmaceutical dosage unit. In some embodiments, at least one excipient other than water is selected from taste-masking agents and sweeteners.

Other uses and advantages of the present invention will be apparent to the person skilled in the art after having considered the description, including the drawings and claims, herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the effect of various active compounds extracted from Scutellaria barbata D. Don on the reactive oxygen species (ROS) generation, as measured by DCFDA fluorescence.

FIG. 2 shows the effect of various active compounds extracted from Scutellaria barbata D. Don on reactive oxygen species (ROS) generation, as measured by dihydroethidium (HE) fluorescence.

FIG. 3 shows the effect of various active compounds extracted from Scutellaria barbata D. Don on mitochondrial reactive oxygen species (ROS) generation, as measured by MitoSOX fluorescence.

FIG. 4 shows the effect of various active compounds extracted from Scutellaria barbata D. Don on the generation of comets in treated cells.

FIG. 5 shows the effect of various active compounds extracted from Scutellaria barbata D. Don on the ATP generation in treated cells.

### DETAILED DESCRIPTION OF THE INVENTION

This invention relates to pharmaceutical compositions and unit dosages that contain active agents isolated from an extract of Scutellaria barbata, at to the methods of using those extracts for the treatment of cancer. In specific embodiments, the herb from which the active compounds are isolated is selected from the species of Scutellaria barbata D. Don of the Labiatae Family.

Additionally, this invention relates to methods of using extracts of Scutellaria barbata D. Don, whereby the extract of Scutellaria barbata D. Don is administered to a patient at heretofore uncharacterized dosages.

The invention further relates to administration of extracts of Scutellaria barbata D. Don, active agents and combinations of active agents derived from extracts of Scutellaria barbata D. Don, especially water extracts of Scutellaria barbata D. Don.

The inventor has found that an extract of Scutellaria barbata D. Don is well-tolerated at doses much higher than previously reported, e.g. at least about 20 g/day of soluble material extracted from Scutellaria barbata D. Don may be administered to a patient without inducing any dose-limiting toxicities. The inventor has administered 20 g/day, 30 g/day, and 40 g/day of soluble matter extracted from Scutellaria barbata D. Don to breast cancer patients without reaching the maximum tolerated dose. Thus, the inventor has identified a dose of at least about 20 g/day, and particularly from about 20 g/day to about 200 g/day, as being within the scope of the present invention. Furthermore, the inventor has found that the extract of Scutellaria barbata D. Don may be conveniently provided in a dosage unit suitable for administration to a patient. Thus, in some embodiments, there is provided a dosage unit comprising at least about 20 g of soluble matter extracted from Scutellaria barbata D. Don. In some embodiments, the unit dose further comprises at least one excipient, especially at least one excipient other than water, and in particular at least one taste-masking agent, sweetener or both. In particular embodiments, the dosage unit is in an form suitable for oral administration, e.g. an aqueous (water-based) composition or a dry powder suitable for reconstitution with water. The inventor has found that the dosage unit is suitable for administration to a cancer patient, especially a cancer patient suffering from breast cancer or a gynecological cancer, such as uterine cancer. In particular embodiments, the dosage unit comprises about 20 g to about 200 g of soluble matter extracted from Scutellaria barbata D. Don. In some embodiments, the dosage unit comprises about 20 g-100 g, about 20 g-60 g, about 20 g-50 g, about 20 g-40 g, about 20 g, about 30 g, about 40 g, about 50 g, about 60 g, or about 40 g-about 100 g of soluble matter extracted from Scutellaria barbata D. Don. The inventor having also identified the anti-cancer active compounds of Scutellaria barbata D. Don (i.e. Luteolin, Apigenin, Scutellarein, and Scutellarin), and their concentrations in the soluble matter extracted from Scutellaria barbata D. Don, the invention also provides a dosage unit at least about 0.25 g of a combination of Luteolin, Apigenin, Scutellarein, and Scutellarin. Some embodiments provide a dosage unit at least about 0.27 g, at least about 0.35 g, about 0.35 g-4 g, about 0.35 g-2 g, about 0.35 g-1.1 g, about 0.35 g to about 1 g, or about 0.35 g to about 0.8 g of the combination of Luteolin, Apigenin, Scutellarein, and Scutellarin.

The inventor having determined that a dose of at least about 20 g per day of soluble matter extracted from Scutellaria barbata D. Don is well-tolerated and effective for the treatment of cancer, the invention further provides a method of treating cancer, comprising administering to a cancer patient at least about 20 g per day of soluble matter extracted from Scutellaria barbata D. Don. In some embodiments, the cancer is selected from breast cancer and one or more gynecological cancers. In some embodiments, said cancer is a breast cancer. In some embodiments, the breast cancer is advanced breast cancer, metastatic breast cancer, treatment-refractory breast cancer, ER-negative breast cancer, PR-negative breast cancer, HER2-negative breast cancer, and/or triple-negative breast cancer. In some embodiments, the method includes administering to the patient about 20 g per day to about 200 g per day of soluble matter extracted from Scutellaria barbata D. Don. In some embodiments, the patient is given about 20 g per day-100 g per day, about 20 g per day-60 g per day, about 20 g per day-50 g per day, about 20 g per day-40 g per day, or about 40 g per day-100 g per day of soluble matter extracted from Scutellaria barbata D. Don. In some embodiments, the soluble matter extracted from Scutellaria barbata D. Don comprises at least about 0.25 g of a combination of Luteolin, Apigenin, Scutellarein, and Scutellarin. In some embodiments, the soluble matter extracted from Scutellaria barbata D. Don comprises at least about 0.27 g of the combination of Luteolin, Apigenin, Scutellarein, and Scutellarin. In some embodiments, the soluble matter extracted from Scutellaria barbata D. Don comprises at least about 0.35 g of the combination of Luteolin, Apigenin, Scutellarein, and Scutellarin. In some embodiments, the soluble matter extracted from Scutellaria barbata D. Don comprises about 0.35 g-4 g, about 0.35 g-2 g, about 0.35 g-1.1 g, about 0.35 g-1 g, about 0.35 g-0.8 g, about 0.35-0.75 g, or about 0.7 g-2 g of the combination of Luteolin, Apigenin, Scutellarein, and Scutellarin.

The inventor has also determined that addition of an excipient, such as a taste-masking agent, to a high dose of a pharmaceutical composition comprising an extract of Scutellaria barbata D. Don attenuates the bitter taste of the extract. As the inventor has found that high doses of Scutellaria barbata D. Don (e.g. at least about 1 g soluble matter per dose or per day) are well-tolerated, but relatively unpalatable, the inventor has found the addition of a taste-masking agent or other agent is desirable for making the composition palatable for consumption of high dosages of Scutellaria barbata D. Don extract, such as for the treatment of cancer. Thus, embodiments described herein provide a pharmaceutical composition (e.g. for the treatment of cancer, especially breast or gynecological cancer) comprising at least one excipient other than water (such as at least one taste-masking agent, sweetener or both), and one or more members of the group consisting of Luteolin, Apigenin, Scutellarein, and Scutellarin, which are the anti-cancer actives that the inventor has identified as being necessary for the anti-cancer activity of an extract of Scutellaria barbata D. Don. The inventor has also found that high molecular weight compounds, e.g. compounds with molecular weights greater than 1000 g/mol (although other cut-offs, such as 1,000-10,000 g/mol may also be used), add to the bulk of the composition without conferring any substantial activity, and tend to cause stomach upset, bloating and/or diarrhea. Thus, in some embodiments, the pharmaceutical composition is depleted of high molecular weight compounds, and in some embodiments is substantially free of high molecular weight compounds. In some embodiments, the composition contains a combination of Luteolin, Apigenin, Scutellarein, and Scutellarin, containing about 1 part Luteolin, about 1.1 parts Apigenin, About 4.8 parts Scutellarein and about 34 parts Scutellarin. (All “parts” determined by weight.) In some embodiments, the combination of Luteolin, Apigenin, Scutellarein, and Scutellarin contains about 1 part Luteolin, about 0.9 to about 1.3 part Apigenin, about 3.9 to about 6 parts Scutellarein, and about 37 to about 43 parts Scutellarin. In some embodiments, the combination of Luteolin, Apigenin, Scutellarein, and Scutellarin contains about 1 part Luteolin, about 0.75 to about 1.64 parts Apigenin, about 3.1 to about 7.5 parts Scutellarein, and about 20.4 to about 54.7 parts Scutellarin. In some embodiments, the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin. In some embodiments, the composition contains about 1 g to about 200 g soluble matter, of which soluble matter about 1% to about 99% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin. In some embodiments, the composition contains about 1 g to about 200 g soluble matter, of which soluble matter about 1% to about 3% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin. In some embodiments, the composition contains about 1 g to about 200 g soluble matter, of which soluble matter about 1.5% to about 2.1% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin. In some embodiments, the composition contains about 1 g to about 200 g soluble matter, of which soluble matter about 1% to about 99% is active soluble matter, of which active soluble matter about 1.9% to about 3% is Luteolin, about 2.2% to about 3.2% is Apigenin, about 9.2% to about 14.5% is Scutellarein, and about 60% to the balance of active soluble matter is Scutellarin. In some embodiments, the composition contains about 1 g to about 200 g soluble matter, of which soluble matter about 1% to about 3% is active soluble matter, of which active soluble matter about 1.9% to about 3% is Luteolin, about 2.2% to about 3.2% is Apigenin, about 9.2% to about 14.5% is Scutellarein, and about 60% to the balance of active soluble matter is Scutellarin. In some embodiments, the composition contains about 1 g to about 200 g soluble matter, of which soluble matter about 1.5% to about 2.1% is active soluble matter, of which active soluble matter about 1.9% to about 3% is Luteolin, about 2.2% to about 3.2% is Apigenin, about 9.2% to about 14.5% is Scutellarein, and about 60% to the balance of active soluble matter is Scutellarin. In some embodiments, the composition contains about 1 g to about 200 g soluble matter, of which soluble matter about 1% to about 50% is active soluble matter, of which active soluble matter about 2.2% to about 2.7% is Luteolin, about 2.4% to about 2.9% is Apigenin, about 10.5% to about 13.2% is Scutellarein, and about 72% to the balance of active soluble matter is Scutellarin. In some embodiments, the composition contains about 1 g to about 200 g soluble matter, of which soluble matter about 1% to about 3% is active soluble matter, of which active soluble matter about 2.2% to about 2.7% is Luteolin, about 2.4% to about 2.9% is Apigenin, about 10.5% to about 13.2% is Scutellarein, and about 72% to the balance of active soluble matter is Scutellarin. In some embodiments, the composition contains about 1 g to about 200 g soluble matter, of which soluble matter about 1.5% to about 2.1% is active soluble matter, of which active soluble matter about 2.2% to about 2.7% is Luteolin, about 2.4% to about 2.9% is Apigenin, about 10.5% to about 13.2% is Scutellarein, and about 72% to the balance of active soluble matter is Scutellarin. In some embodiments, the composition is used to treat a breast cancers selected from one or more of is advanced breast cancer, metastatic breast cancer, treatment-refractory breast cancer, ER-negative breast cancer, PR-negative breast cancer, HER2-negative breast cancer, and/or triple-negative breast cancer.

Some embodiments described herein provide a method of treating cancer, especially one or more breast and/or gynecological cancers, comprising administering to the patient an effective amount of a composition comprising at least one excipient other than water (such as at least one taste-masking agent, sweetener or both), and one or more members of the group consisting of Luteolin, Apigenin, Scutellarein, and Scutellarin. In some embodiments, the composition is used to treat a breast cancers selected from one or more of is advanced breast cancer, metastatic breast cancer, treatment-refractory breast cancer, ER-negative breast cancer, PR-negative breast cancer, HER2-negative breast cancer, and/or triple-negative breast cancer. In some embodiments, the effective amount of the composition comprises at least 0.25 g of a combination of Luteolin, Apigenin, Scutellarein, and Scutellarin. In some embodiments, the effective amount of the composition comprises at least 0.27 g of a combination of Luteolin, Apigenin, Scutellarein, and Scutellarin. In some embodiments, the composition comprises at least about 0.35 g of the combination of Luteolin, Apigenin, Scutellarein, and Scutellarin. In some embodiments, the effective amount of the composition contains about 0.27 g to about 4 g of the combination of Luteolin, Apigenin, Scutellarein, and Scutellarin. In some embodiments, the effective amount of the composition contains about 0.35 g to about 4 g of the combination of Luteolin, Apigenin, Scutellarein, and Scutellarin. In some embodiments, the effective amount of the composition contains about 0.35 g-2 g, about 0.35 g-1.1 g, about 0.35-1 g, about 0.35 g to about 0.8 g of the combination of Luteolin, Apigenin, Scutellarein, and Scutellarin. In some embodiments, the combination of Luteolin, Apigenin, Scutellarein, and Scutellarin contains: about 1 part Luteolin, about 1.1 parts Apigenin, About 4.8 parts Scutellarein and about 34 parts Scutellarin; about 1 part Luteolin, about 0.9 to about 1.3 part Apigenin, about 3.9 to about 6 parts Scutellarein, and about 37 to about 43 parts Scutellarin; about 1 part Luteolin, about 0.75 to about 1.64 parts Apigenin, about 3.1 to about 7.5 parts Scutellarein, and about 20.4 to about 54.7 parts Scutellarin; about 1 part Luteolin, about 0.61 to about 2 parts Apigenin, about 2.5 to about 9.4 parts Scutellarein, and about 15 to about 70 parts Scutellarin; about 6.7 mg to about 90 mg of Luteolin, about 8.9 mg to about 90 mg of Luteolin, about 8.9 mg to about 50 mg of Luteolin, about 8.9 mg to about 30 mg of Luteolin, about 8.9 mg to about 25 mg of Luteolin, or about 8.9 mg to about 20 mg of Luteolin; about 7.3 mg to about 100 mg of Apigenin, about 9.7 mg to about 100 mg of Apigenin, about 9.7 mg to about 50 mg of Apigenin, about 9.7 mg to about 30 mg of Apigenin, about 9.7 mg to about 25 mg of Apigenin, or about 9.7 mg to about 20 mg of Apigenin; about 30 mg to about 500 mg of Scutellarein, about 40 mg to about 500 mg of Scutellarein, about 40 mg to about 220 mg of Scutellarein, about 40 mg to about 130 mg of Scutellarein, about 40 mg to about 110 mg of Scutellarein, or about 40 mg to about 90 mg of Scutellarein; about 0.25 g to about 3 g of Scutellarin, about 0.3 g to about 3 g of Scutellarin, about 0.3 g to about 1.5 g of Scutellarin, about 0.3 g to about 0.9 g of Scutellarin, about 0.3 g to about 0.8 g of Scutellarin, or about 0.3 g to about 0.65 g of Scutellarin.

The inventor has found that a combination of Luteolin, Apigenin, Scutellarein, and Scutellarin is effective as a treatment for cancer, especially breast cancer. Thus, in some embodiments the invention provides a dosage unit comprising a combination of Luteolin, Apigenin, Scutellarein, and Scutellarin. In some embodiments, the dosage unit comprises at least about 0.25 g of a combination of Luteolin, Apigenin, Scutellarein, and Scutellarin. In some embodiments, the dosage unit comprises at least about 0.27 g, or at least about 0.35 g of a combination of Luteolin, Apigenin, Scutellarein, and Scutellarin. In some embodiments, the dosage unit comprises about 0.35 g to about 4 g, about 0.35 g to about 2 g, about 0.35 g to about 1.1 g, about 0.35 g to about 1 g, about 0.35 g to about 0.8 g, or about 0.7 g to about 2 g of the combination of Luteolin, Apigenin, Scutellarein, and Scutellarin. In some embodiments, the dosage unit further comprises at least one excipient other than water, such as a taste masking agent, a sweetener or both. In some embodiments, the dosage unit is substantially free of high molecular weight compounds extracted from Scutellaria barbata D. Don. In some embodiments, the combination of Luteolin, Apigenin, Scutellarein, and Scutellarin contains: about 1 part Luteolin, about 1.1 parts Apigenin, About 4.8 parts Scutellarein and about 34 parts Scutellarin; about 1 part Luteolin, about 0.9 to about 1.3 part Apigenin, about 3.9 to about 6 parts Scutellarein, and about 37 to about 43 parts Scutellarin; about 1 part Luteolin, about 0.75 to about 1.64 parts Apigenin, about 3.1 to about 7.5 parts Scutellarein, and about 20.4 to about 54.7 parts Scutellarin; about 1 part Luteolin, about 0.61 to about 2 parts Apigenin, about 2.5 to about 9.4 parts Scutellarein, and about 15 to about 70 parts Scutellarin; about 6.7 mg to about 90 mg of Luteolin, about 8.9 mg to about 90 mg of Luteolin, about 8.9 mg to about 50 mg of Luteolin, about 8.9 mg to about 30 mg of Luteolin, about 8.9 mg to about 25 mg of Luteolin, or about 8.9 mg to about 20 mg of Luteolin; about 7.3 mg to about 100 mg of Apigenin, about 9.7 mg to about 100 mg of Apigenin, about 9.7 mg to about 50 mg of Apigenin, about 9.7 mg to about 30 mg of Apigenin, about 9.7 mg to about 25 mg of Apigenin, or about 9.7 mg to about 20 mg of Apigenin; about 30 mg to about 500 mg of Scutellarein, about 40 mg to about 500 mg of Scutellarein, about 40 mg to about 220 mg of Scutellarein, about 40 mg to about 130 mg of Scutellarein, about 40 mg to about 110 mg of Scutellarein, or about 40 mg to about 90 mg of Scutellarein; about 0.25 g to about 3 g of Scutellarin, about 0.3 g to about 3 g of Scutellarin, about 0.3 g to about 1.5 g of Scutellarin, about 0.3 g to about 0.9 g of Scutellarin, about 0.3 g to about 0.8 g of Scutellarin, or about 0.3 g to about 0.65 g of Scutellarin. In some embodiments, the compositions are employed in the treatment of cancer, such as breast cancer and/or one or more gynecological cancers. In some embodiments, the cancer is a breast cancer, such as advanced breast cancer, metastatic breast cancer, treatment-refractory breast cancer, ER-negative breast cancer, PR-negative breast cancer, HER2-negative breast cancer, and/or triple-negative breast cancer.

The inventor has further discovered processes for making pharmaceutical compositions using the aerial portions of Scutellaria barbata D. Don as starting materials. Such processes are particularly useful for making compositions comprising Luteolin, Apigenin, Scutellarein, and Scutellarin. Thus, in some embodiments, the inventor has described a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; (d) optionally evaporating some, substantially all or all of the water from the refined extract or adding additional water to the refined extract; and (e) combining the refined extract with at least one pharmaceutically acceptable excipient other than water, to form the pharmaceutical composition. In some embodiments, the refined extract contains Apigenin, Luteolin, Scutellarein, and Scutellarin. In some embodiments, at least one pharmaceutically acceptable excipient other than water is selected from taste masking agents and sweeteners.

The inventor has found that removing at least some of the high molecular weight compounds extracted from Scutellaria barbata D. Don improves the clinical characteristics of the pharmaceutical composition. As a large amount of soluble matter extracted from Scutellaria barbata D. Don is inactive, reducing the amount of soluble matter by removing molecules having molecular weights above a predetermined cutoff will greatly reduce the bulk of a pharmaceutical composition derived from an extract of Scutellaria barbata D. Don. Additionally, a large part of the soluble matter extracted from Scutellaria barbata D. Don into water is soluble fiber, which is not absorbed in the intestines and tends to promote gastrointestinal upset, bloating, gas and diarrhea. Thus, removing at least part of the soluble fiber by reducing the burden of soluble matter extracted from Scutellaria barbata D. Don, while preserving the mixture of Luteolin, Apigenin, Scutellarein, and Scutellarin in the composition, results in an improved anti-cancer drug. Thus, in some embodiments, the invention provides a pharmaceutical composition comprising 1 part of a combination of Luteolin, Apigenin, Scutellarein, and Scutellarin and less than about 50 parts of high molecular weight compounds having molecular weights greater than a predetermined cutoff, wherein the predetermined cutoff is from 1,000 grams/mole to about 20,000 grams/mole. In some embodiments, the pharmaceutical composition comprises about 1 part of the combination of Luteolin, Apigenin, Scutellarein, and Scutellarin and less than about 40 parts, less than about 30 parts, less than about 20 parts, less than about 10 parts, less than about 5 parts, less than about 2 parts, less than about 1 part, less than about 0.5 parts, about 0.01 to about 40 parts, about 0.01 to about 20 parts or about 0.01 to about 10 parts of the high molecular weight compounds. In some embodiments, the cutoff for the high molecular weight compounds is: 10,000 grams/mole; 5,000 grams/mole; 2,000 grams/mole; 1,000 grams/mole; or in a range of about 1,000 grams/mole to about 10,000 grams/mole; about 1,000 grams/mole to about 5,000 grams/mole or about 1,000 grams/mole to about 2,000 grams/mole. In some embodiments, the pharmaceutical composition comprises at least one excipient other than water. In some embodiments, at least one excipient other than water is a taste masking agent, a sweetener or both. The inventor has found that the pharmaceutical composition described herein is conveniently prepared as a dosage unit comprising a pharmaceutical composition described herein. In some embodiments, the dosage unit comprises at least about 18 mg of a combination of Luteolin, Apigenin, Scutellarein, and Scutellarin. In some embodiments, the dosage unit comprises: about 0.25 g to about 4 g of the combination of Luteolin, Apigenin, Scutellarein, and Scutellarin; about 0.27 g to about 4 g of the combination of Luteolin, Apigenin, Scutellarein, and Scutellarin; about 0.35 to about 4 g of the combination of Luteolin, Apigenin, Scutellarein, and Scutellarin; about 0.35 to about 2 g of the combination of Luteolin, Apigenin, Scutellarein, and Scutellarin; about 0.35 to about 1.1 g of the combination of Luteolin, Apigenin, Scutellarein, and Scutellarin; about 0.35 to about 1 g of the combination of Luteolin, Apigenin, Scutellarein, and Scutellarin; about 0.35 to about 0.8 g of the combination of Luteolin, Apigenin, Scutellarein, and Scutellarin; or about 0.7 to about 2 g of the combination of Luteolin, Apigenin, Scutellarein, and Scutellarin. In some embodiments, in addition to the combination of Luteolin, Apigenin, Scutellarein, and Scutellarin, the composition further comprises at least one excipient other than water. In some embodiments, at least one excipient other than water is selected from taste masking agents, sweeteners, or both. In some embodiments, the invention provides method of treating cancer comprising administering to a cancer patient an effective amount of a pharmaceutical composition or a dosage form described herein. In some embodiments, the cancer is one or more breast cancers and/or gynecological cancers, such as breast or uterine cancer. In some embodiments, is a breast cancer, such as advanced breast cancer, metastatic breast cancer, treatment-refractory breast cancer, ER-negative breast cancer, PR-negative breast cancer, HER2-negative breast cancer, and/or triple-negative breast cancer.

The inventor has also discovered a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; (d) optionally evaporating some, substantially all or all of the water from the refined extract or adding additional water to the refined extract; and (e) combining the refined extract with a pharmaceutically acceptable excipient to form the pharmaceutical composition. 1 part Luteolin, about 0.61 to about 2 parts Apigenin, about 2.5 to about 9.4 parts Scutellarein, and about 15 to about 70 parts Scutellarin.

Some embodiments also provide a process of making a refined extract of Scutellaria barbata D. Don, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form the refined extract of Scutellaria barbata D. Don.

Some embodiments further provide a process of making a pharmaceutical composition, comprising combining at least one pharmaceutically acceptable excipient other than water with one or more members of the group consisting of Luteolin, Apigenin, Scutellarein, and Scutellarin to form the pharmaceutical composition. In some embodiments, at least one pharmaceutical excipient other than water is selected from taste masking agents and sweeteners.

Some embodiments provide a process of making a pharmaceutical dosage unit comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form a refined extract; and (d) combining the refined extract with at least one excipient other than water to form the pharmaceutical dosage unit. In some embodiments, at least one excipient other than water is selected from taste-masking agents and sweeteners.

The term “about” followed by a stated value is intended to indicate a value within the range of experimental error (generally within one standard deviation) of the stated value. Unless the experimental error is specifically determined, the term “about” followed by a stated value “x” may be taken to mean X±0.1X.

Processes for the Manufacture of Pharmaceutical Compositions and Unit Dosages Containing Scutellaria barbata Extracts

The pharmaceutical compositions and unit dosages described herein contain soluble matter (i.e. matter that is soluble in water) that is extracted from Scutellaria barbata, specifically the aerial parts of Scutellaria barbata D. Don. Herba Scutellaria barbata D. Don (Lamiaceae) of the Labiatae family—Ban Zhi Lian (BZL) is grown mainly, though not exclusively, in areas southeastern of the Yellow River (Huang Po) in the provinces of Sichuan, Jiangsu, Jiangxi, Fujian, Guangdong, Guangxi and Shaanxi. The plant is harvested in late summer and early autumn after it blooms (May-June). The aerial part is cut from the root. Only the aerial parts (leaves and stems) are used for the preparation of compositions and dosage units described herein.

Table 1 depicts nomenclature for the herb, Scutellaria barbata D. Don, from which extracts of this invention are obtained, listed by family, genus, species and tradition Chinese name, of this invention.

TABLE 1

| Family | genus | Species | Chinese name | Herb part |
|---|---|---|---|---|
| Labiatae | Scutellaria | barbata D. Don | Ban Zhi Lian | aerial |

Pharmaceutical Compositions

Some embodiments described herein provide pharmaceutical compositions, especially pharmaceutical compositions for the treatment of cancer. In particular, the invention provides pharmaceutical compositions (“compositions”) for treatment of gynecological cancers and breast cancer. In some preferred embodiments, the compositions are for the treatment of breast cancer, especially those breast cancers that have been considered by oncologists to be especially difficult to treat, which are described in greater detail below, but which include: advanced breast cancer, metastatic breast cancer, breast cancer that is negative for one or more hormone receptors (e.g. ER-negative, PR-negative, and/or HER2-negative breast cancers), and breast cancers that have been unsuccessfully treated previously with one or more cancer therapies, such as radiation therapy, proton therapy, and/or chemotherapy. The inventor has found that treatment of a patient having one or more of these types of cancer with at least about 20 g soluble matter extracted from Scutellaria barbata D. Don is well-tolerated and effective in the treatment of these cancers.

In some embodiments, the invention provides a pharmaceutical composition comprising at least one excipient other than water, and one or more members of the group consisting of Luteolin, Apigenin, Scutellarein, and Scutellarin. It has been found that, though each of Luteolin, Apigenin, Scutellarein, and Scutellarin possesses activity which, on a molecular level, indicates anti-cancer activity, the combination of all four of these compounds is particularly potent against cancer, particularly breast cancer, even breast cancer that has proven refractory to prior treatment. Thus, in some preferred embodiments, the composition comprises each of Luteolin, Apigenin, Scutellarein, and Scutellarin.

It has also been found that, especially at the higher doses used in the methods of treating cancer described herein, extracts of Scutellaria barbata D. Don can be unpalatable, even to the point of discouraging patient compliance. Accordingly, it is desired to use a taste-masking agent, such as a flavoring or other taste-masking agent, a sweetener, or both, to make the compositions more palatable, thereby enhancing patient comfort with the treatment, and potentially enhancing patient compliance. Thus, in some embodiments, at least one excipient other than water is selected from taste masking agents and sweeteners. As used herein, to say that a pharmaceutical composition contains, comprises or otherwise includes “an excipient other than water” means that the composition must contain some excipient aside from water, though it may also contain water, if water is an appropriate excipient for the particular form of the dosage unit in which the pharmaceutical composition is present. Some embodiments, for example, include soluble matter extracted from Scutellaria barbata D. Don, a taste masking agent, and water. Other embodiments may further include a sweetener in addition to the taste masking agent, or may employ a sweetener instead of the taste masking agent.

It has also been discovered by the inventor that high doses of Scutellaria barbata D. Don extracts (e.g. at least 20 g soluble matter extracted from Scutellaria barbata D. Don or higher) cause stomach upset, bloating, gas and/or diarrhea in at least some patients. The inventor has determined that high molecular weight compounds extracted from Scutellaria barbata D. Don are inactive against breast and/or gynecological cancers and tend to induce gastrointestinal distress, especially at doses of, or exceeding, 20 g/day. At the doses described herein, such stomach discomfort could, at least for some patients, result in poor patient compliance or even discontinuance of therapy. By removing at least some of the high molecular weight compounds from the soluble matter extracted from Scutellaria barbata D. Don (e.g. by nanofiltration), it is contemplated that the bulk amount of soluble matter that must be administered to patients will be reduced, and the gastrointestinal discomfort associated with high concentrations of soluble matter extracted from Scutellaria barbata D. Don will be reduced. Thus, the inventor herein provides teaching of compositions that are depleted, and in some cases substantially free, of high molecular weight compounds.

A combination of Luteolin, Apigenin, Scutellarein, and Scutellarin may otherwise be referred to herein as “active soluble matter” as opposed to “inactive soluble matter”, which includes “high molecular weight compounds” as well as compounds that are not high molecular weight compounds but are not active in the treatment of breast and/or gynecological cancers. Thus, the mass of soluble matter is equal to the sum of masses of active soluble matter (Luteolin, Apigenin, Scutellarein, and Scutellarin) and inactive soluble matter. The mass of inactive soluble matter is the sum of the masses of high molecular weight compounds and other inactive compounds.

As used herein “high molecular weight compounds” refers to those compounds that are co-extracted with Luteolin, Apigenin, Scutellarein, and Scutellarin during the process of water extraction of Scutellaria barbata D. Don, and that have molecular weights of, or greater than, a predetermined cut-off. In some embodiments, the cut-off may be somewhere from 1,000 g/mol to about 10,000 g/mol. In some embodiments, the cutoff of 10,000 grams per mole will suffice to remove a high percentage of soluble fiber from the soluble extract of Scutellaria barbata D. Don; however, lower cut-offs are contemplated and are, in some cases, preferred, as lower cutoffs will allow achievement of greater concentrations of Luteolin, Apigenin, Scutellarein, and Scutellarin in the pharmaceutical compositions and dosage units, and will reduce the bulk of soluble matter that must be administered to patients to achieve a therapeutic effect. In some embodiments, the cut-off is in the range of 750-20,000 g/mol, preferably in the range of 750-10,000 g/mol, and more particularly 750-5,000 g/mol. Particular cut-offs include: 750 g/mol; 1,000 g/mol; 2,000 g/mol; 5,000 g/mol; and 10,000 g/mol.

Thus, some embodiments of compositions and dosage units provided herein are substantially free of high molecular weight compounds. The term “substantially free” as used herein means that the composition or dosage unit contains less than some predetermined fraction of high molecular weight compounds than were contained in a “crude extract,” which is a water extract of aerial parts of Scutellaria barbata D. Don that been treated (e.g. filtered or decanted) to remove insoluble matter (e.g. stems, leaves and insoluble portions thereof) but has not been otherwise treated to remove high molecular weight compounds. In some embodiments, the predetermined fraction is 1/10 (0.1), 1/20 (0.05), 1/50 (0.02), 1/100 (0.01), 1/200 (0.005), 1/500 (0.002) or 1/1000 (0.001). Particular values for “substantially free of high molecular weight compounds” can also be expressed relative to the total mass of soluble matter extracted from Scutellaria barbata D. Don contained in the pharmaceutical composition. In some embodiments, a composition that is substantially free of high molecular weight compounds contains less than about 10 wt %, less than about 5 wt %, less than about 1 wt %, less than about 0.5 wt % or less than about 0.1 wt % of high molecular weight compounds relative to the total amount of soluble matter extracted from Scutellaria barbata D. Don. Particular values for “substantially free of high molecular weight compounds” further be expressed as a mass proportion relative to the amount of Luteolin, Apigenin, Scutellarein, and Scutellarin contained in the composition. In some embodiments, about 1% to about 99% of soluble matter extracted from Scutellaria barbata D. Don in the pharmaceutical composition is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin

In some cases, it may be sufficient to remove only part of the high molecular weight compounds from the soluble matter extracted from Scutellaria barbata D. Don. Thus, in some embodiments of compositions and dosage units provided herein are depleted of high molecular weight compounds. The term “depleted” as used herein means that the composition or dosage unit contains less than some predetermined fraction of high molecular weight compounds than were contained in a “crude extract,” which is described in the previous paragraph. In some embodiments, the predetermined fraction is 9/10 (0.9), 8/10 (0.8), 7/10 (0.7), 6/10 (0.6), ½ (0.5), ⅓ (0.333) or ¼ (0.25). Particular values for “depleted of high molecular weight compounds” can also be expressed relative to the total mass of soluble matter extracted from Scutellaria barbata D. Don contained in the pharmaceutical composition. In some embodiments, a composition that is depleted of high molecular weight compounds contains less than about 90 wt %, less than about 80 wt %, less than about 70 wt %, less than about 60 wt % or less than about 50 wt % of high molecular weight compounds relative to the total amount of soluble matter extracted from Scutellaria barbata D. Don. Particular values for “depleted of high molecular weight compounds” further be expressed as a mass proportion relative to the amount of Apigenin, Luteolin, Scutellarein and Scutellarin contained in the composition. In some embodiments, about 1% to about 99% of soluble matter extracted from Scutellaria barbata D. Don in the pharmaceutical composition is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin.

It has been found that the active compounds (Luteolin, Apigenin, Scutellarein, and Scutellarin) extracted from Scutellaria barbata D. Don tend to be represented in the water extracts of aerial parts of Scutellaria barbata D. Don in certain characteristic proportions that appear to be critical to their combined activity. In some embodiments, the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin. In some embodiments, the combination of Luteolin, Apigenin, Scutellarein, and Scutellarin contains about 1 part Luteolin, about 0.75 to about 1.64 parts Apigenin, about 3.1 to about 7.5 parts Scutellarein, and about 20.4 to about 54.7 parts Scutellarin. In some embodiments, the combination of Luteolin, Apigenin, Scutellarein, and Scutellarin contains about 1 part Luteolin, about 0.9 to about 1.3 part Apigenin, about 3.9 to about 6 parts Scutellarein, and about 37 to about 43 parts Scutellarin. In some embodiments, the composition contains a combination of Luteolin, Apigenin, Scutellarein, and Scutellarin, containing about 1 part Luteolin, about 1.1 parts Apigenin, About 4.8 parts Scutellarein and about 34 parts Scutellarin.

Processes of Making Pharmaceutical Compositions

The pharmaceutical compositions provided herein may be produced by a process that includes extracting active compounds from aerial parts (stems and/or leaves) of Scutellaria barbata D. Don, e.g. with water. As described herein with reference to extraction, “water” includes pure water (e.g. water for injection, distilled water, double deionized water, filtered distilled water, etc.) as well as aqueous solutions that consist of water and one or more minor solid or liquid solutes, so long as the majority of the extraction medium is water and the solute or solutes do not materially affect the extraction properties of water. In some preferred embodiments, the process also includes removing a portion of high molecular weight compounds from the extract of Scutellaria barbata D. Don, as described in more detail above.

Thus, in some embodiments, there is provided a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; and (e) combining the refined extract with at least one pharmaceutically acceptable excipient other than water, to form the pharmaceutical composition. In some embodiments, the process also includes (d) optionally evaporating some, substantially all or all of the water from the refined extract or adding additional water to the refined extract. In some embodiments, at least one pharmaceutically acceptable excipient other than water is selected from taste masking agents and sweeteners. In some embodiments, the pharmaceutical composition may be further combined with suitable packaging to form a suitable dosage unit.

The aerial parts of Scutellaria barbata D. Don (leaves and/or stems) are combined with water and heated to a suitable temperature above room temperature, especially about 40° C., and more preferably from about 50° C. to about 80° C., optionally at elevated pressures. The mixture should be cooked long enough to extract the active compounds into the aqueous phase of the mixture, but not so long as to unnecessarily waste energy or cause breakdown in the active compounds. Some period longer than about 10 minutes, but less than about 2 days is suitable, though periods of 30 minutes to 6 hours are generally considered suitable. More particular values are recited in the examples herein.

Once cooked, the aerial portions of Scutellaria barbata D. Don are separated from the aqueous phase by some suitable method. Larger parts may be removed by straining the mixture through a sieve, whereas smaller parts may be removed by filtration. The filtration may be performed in stages, with each stage involving passage through one or more filters of successively smaller pore size.

High molecular weight compounds may be removed by a suitable method, such as nanofiltration or size exclusion chromatography.

Optionally, the volume of the solution may be reduced, e.g. by evaporating off part of the water. The solution may also be freeze dried or otherwise desiccated to form a dry residue, which may be pulverized to form a powder. In any case, the resulting refined extract can then be combined with at least one excipient, especially an excipient other than water, to form the pharmaceutical composition. In some embodiments, the excipient other than water is a taste masking agent or a sweetener. In some preferred embodiments, the excipient other than water contains a taste masking agent.

Other embodiments provide a process of making a pharmaceutical composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; (c) separating high molecular weight compounds from the crude extract to form a refined extract; and (e) combining the refined extract with a pharmaceutically acceptable excipient to form the pharmaceutical composition. Some embodiments also include (d) optionally evaporating some, substantially all or all of the water from the refined extract or adding additional water to the refined extract. In some embodiments, the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin. In some embodiments, the combination of Luteolin, Apigenin, Scutellarein, and Scutellarin contains about 1 part Luteolin, about 0.75 to about 1.64 parts Apigenin, about 3.1 to about 7.5 parts Scutellarein, and about 20.4 to about 54.7 parts Scutellarin. In some embodiments, the combination of Luteolin, Apigenin, Scutellarein, and Scutellarin contains about 1 part Luteolin, about 0.9 to about 1.3 part Apigenin, about 3.9 to about 6 parts Scutellarein, and about 37 to about 43 parts Scutellarin. In some embodiments, the composition contains a combination of Luteolin, Apigenin, Scutellarein, and Scutellarin, containing about 1 part Luteolin, about 1.1 parts Apigenin, About 4.8 parts Scutellarein and about 34 parts Scutellarin.

In some embodiments, there is provided a process of making a composition, comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form a refined extract. The refined extract may be further processed to produce a dosage unit as described herein. In some embodiments, the refined extract is depleted of high molecular weight compounds. In some embodiments, the refined extract is substantially free of high molecular weight compounds.

As described above, it is considered desirable in certain circumstances to mask the taste of active compounds contained in extracts of Scutellaria barbata D. Don, especially where the dosage is at least about 20 g/day. Thus, some embodiments the process of making a pharmaceutical composition comprises combining at least one pharmaceutically acceptable excipient other than water (e.g. a taste-masking agent and/or a sweetener) with one or more members of the group consisting of Luteolin, Apigenin, Scutellarein, and Scutellarin to form the pharmaceutical composition.

In some such embodiments, at least one pharmaceutical excipient other than water is selected from taste masking agents and sweeteners.

Dosage Units

It is considered by the current inventor that the pharmaceutical compositions described herein are conveniently prepared in dosage units for convenient distribution, storage and administration. There is a distinction between “dose” and “dosage unit” as described herein. As used herein, the term “dose” refers to an amount of the pharmaceutical composition administered in a single occurrence. A daily dose is an amount of the pharmaceutical composition administered in a day. Doses may be administered once daily (Q.D.), twice-daily (b.i.d.), trice daily (t.i.d.), four times daily (q.i.d.), etc.

As used herein, the term “dosage unit” is a single, pre-manufactured form of the pharmaceutical composition that consists of one or more doses of the pharmaceutical composition, or some fraction of a dose of the pharmaceutical composition that can be combined with other dosage units to form a single dose. In some embodiments, the dosage unit consists of a single day's dose of the pharmaceutical composition. The dosage unit may adapted to be administered as a single daily dose (Q.D.) or may be divided into two, three, four or more doses (b.i.d., t.i.d., or q.i.d., respectively) to be administered at different times of the day, or may be administered as a single dose. (This is especially true of elixirs, which may be divided into two or more doses per dosage unit, as well as tablets, which may be divided into two or more dosage units for administration at different times during a day.) In some other embodiments, the dosage unit may comprise some fraction (e.g. half, a third, a fourth, a fifth) of a single dose. A dosage unit may also be a solution for injection of a particular volume, e.g. 20 mL to 1000 mL, for administration via a drip line or similar intravenous administration method, or even via a nasopharyngeal tube.

Some preferred embodiments of the dosage units include tablets, capsules, powders and solutions (elixirs).

Tablets include tablets to be swallowed, tablets to be chewed and swallowed and tablets adapted to dissolve on the tongue and be swallowed, with or without a liquid swallowing aid, such as water. Suitable excipients for tablets include binding agents, fillers, disintegrants, dispersants, glidants, ant-sticking and anti-caking agents, as well as taste-masking agents and sweeteners.

Capsules include capsules to be swallowed whole as well as capsules adapted to be dissolved in a liquid excipient, such as water. Capsules also include capsules to be opened and their contents dissolved in a suitable excipient, such as water. Suitable excipients for capsules include dispersants, fillers, taste-masking agents and sweeteners.

Powders include powders that have been packaged in a suitable container for transportation and storage, such as a foil pouch, a sealed vial, etc. Suitable excipients for powders include dispersants, fillers, taste-masking agents and sweeteners.

Solutions include water-based solutions containing water, an excipient other than water and active soluble matter extracted from Scutellaria barbata D. Don (Luteolin, Apigenin, Scutellarein, and Scutellarin). In preferred embodiments, solutions are packaged in a suitable sealed container and packaged with instructions for administration of the solution to a patient. For intravenous administration, the water-based solution may or may not contain an excipient other than water.

The inventor has found that compositions described herein should be administered to patients, and importantly can be tolerated by patients, at levels that were heretofore not contemplated. It has surprisingly been found, for example, that compositions as described herein can be administered to patients at high doses, i.e. doses greater than 10 or 12 grams per day of soluble material extracted from Scutellaria barbata D. Don. This administration surprisingly causes no dose limiting toxicities at high doses, especially at doses from 20 grams per day to about 40 grams per day (specifically at 20, 30 and 40 grams per day.) Based on these clinical data, the inventor surmises that the maximum tolerable dose is greater than 40 grams per day, and indeed may be up to about 200 grams per day, more probably up to about 100 grams per day. Thus, some embodiments described herein provide a pharmaceutical dosage unit comprising at least about 20 grams of an active pharmaceutical ingredient that contains at least one member of the group consisting of Apigenin, Luteolin, Scutellarein and Scutellarin. In some embodiments, the active pharmaceutical ingredient contains each of Luteolin, Apigenin, Scutellarein, and Scutellarin. In some preferred embodiments, the dosage unit is an oral dosage unit. In some preferred embodiments, the dosage unit further comprises at least one excipient other than water. In some preferred embodiments, the dosage unit comprises at least one excipient selected from taste masking agents and sweeteners. In some embodiments, the dosage unit comprises at least about 20 grams of the active pharmaceutical ingredient. In some embodiments, the dosage unit is capable of being split between two or more doses for administration in a single day.

In some embodiments, the pharmaceutical dosage unit comprises an active pharmaceutical ingredient containing at least about 20 grams of soluble material extracted from Scutellaria barbata D. Don. The soluble material extracted from Scutellaria barbata D. Don contains one or more of Apigenin, Luteolin, Scutellarein and Scutellarin; preferably it contains all four of Apigenin, Luteolin, Scutellarein and Scutellarin. In particularly preferred embodiments, the soluble material contains each of Apigenin, Luteolin, Scutellarein and Scutellarin in proportions of about: about 1 part Luteolin, about 0.61 to about 2 parts Apigenin, about 2.5 to about 9.4 parts Scutellarein, and about 15 to about 70 parts Scutellarin; about 0.75 to about 1.64 parts Apigenin, about 3.1 to about 7.5 parts Scutellarein, and about 20.4 to about 54.7 parts Scutellarin about 0.75 to about 1.64 parts Apigenin, about 3.1 to about 7.5 parts Scutellarein, and about 20.4 to about 54.7 parts Scutellarin; about 0.9 to about 1.3 part Apigenin, about 3.9 to about 6 parts Scutellarein, and about 37 to about 43 parts Scutellarin; or about 1 part Luteolin, about 1.1 parts Apigenin, About 4.8 parts Scutellarein and about 34 parts Scutellarin. In some embodiments, the soluble material extracted from Scutellaria barbata D. Don is depleted, or substantially free, of high molecular weight compounds. In some embodiments, the dosage unit is an oral dosage unit (e.g. a tablet to be swallowed whole, chewed and swallowed or allowed to dissolve on the tongue and swallowed, a capsule to be swallowed whole, a capsule to be opened and its contents dissolved in a suitable liquid excipient to be swallowed, a capsule to be dissolved whole in a suitable excipient, a powder to be dissolved in a suitable excipient, which may include a taste masking agent, a sweetener, etc. and/or water). Thus, in some embodiments, the dosage unit further comprises at least one excipient other than (e.g. in addition to) water. In some embodiments, the dosage unit comprises at least one excipient selected from taste masking agents and sweeteners. In some embodiments, the dosage unit comprises at least about 20 grams of the active pharmaceutical ingredient (e.g. 20-200 grams per dosage unit, 20-100 grams per dosage unit or 20-60 grams per dosage unit).

In some embodiments, the pharmaceutical dosage unit comprises an active pharmaceutical ingredient containing at least about at least 0.25 g of a combination of Luteolin, Apigenin, Scutellarein, and Scutellarin. In some embodiments, the pharmaceutical dosage unit comprises at least about 0.27 g of Luteolin, Apigenin, Scutellarein, and Scutellarin or at least about 0.35 g of Luteolin, Apigenin, Scutellarein, and Scutellarin. In some embodiments, the pharmaceutical dosage unit comprises about 0.35 g-4 g, 0.35 g-2 g, 0.35 g-1.1 g, 0.35 g-1 g, or 0.35 g-0.8 g of Luteolin, Apigenin, Scutellarein, and Scutellarin. In some embodiments, the pharmaceutical dosage unit comprises about 0.25 g, about 0.27 g, about 0.3 g, about 0.35 g, about 0.4 g, about 0.45 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, of about 4 g of Luteolin, Apigenin, Scutellarein, and Scutellarin. The soluble material extracted from Scutellaria barbata D. Don contains one or more of Apigenin, Luteolin, Scutellarein and Scutellarin; preferably it contains all four of Apigenin, Luteolin, Scutellarein and Scutellarin. In particularly preferred embodiments, the soluble material contains each of Apigenin, Luteolin, Scutellarein and Scutellarin in proportions of about: about 1 part Luteolin, about 0.61 to about 2 parts Apigenin, about 2.5 to about 9.4 parts Scutellarein, and about 15 to about 70 parts Scutellarin; about 0.75 to about 1.64 parts Apigenin, about 3.1 to about 7.5 parts Scutellarein, and about 20.4 to about 54.7 parts Scutellarin about 0.75 to about 1.64 parts Apigenin, about 3.1 to about 7.5 parts Scutellarein, and about 20.4 to about 54.7 parts Scutellarin; about 0.9 to about 1.3 part Apigenin, about 3.9 to about 6 parts Scutellarein, and about 37 to about 43 parts Scutellarin; or about 1 part Luteolin, about 1.1 parts Apigenin, About 4.8 parts Scutellarein and about 34 parts Scutellarin. In some embodiments, the soluble material extracted from Scutellaria barbata D. Don is depleted, or substantially free, of high molecular weight compounds. In some embodiments, the dosage unit is an oral dosage unit (e.g. a tablet to be swallowed whole, chewed and swallowed or allowed to dissolve on the tongue and swallowed, a capsule to be swallowed whole, a capsule to be opened and its contents dissolved in a suitable liquid excipient to be swallowed, a capsule to be dissolved whole in a suitable excipient, a powder to be dissolved in a suitable excipient, which may include a taste masking agent, a sweetener, etc. and/or water). Thus, in some embodiments, the dosage unit further comprises at least one excipient other than (e.g. in addition to) water. In some embodiments, the dosage unit comprises at least one excipient selected from taste masking agents and sweeteners.

The dosage units described herein may be produced by a process according to the invention. In some embodiments, there is provided a process of making a pharmaceutical dosage unit comprising: (a) contacting aerial parts of Scutellaria barbata D. Don with water heated to above 40° C. for a period at least about 10 minutes to form a mixture; (b) separating the aerial parts of Scutellaria barbata D. Don from the mixture to produce a crude extract; and (c) separating high molecular weight compounds from the crude extract to form a refined extract; and (d) combining the refined extract with at least one excipient other than water to form the pharmaceutical dosage unit. In some embodiments, at least one excipient other than water is selected from taste-masking agents and sweeteners. Such dosage units contain a suitable quantity of refined extract to treat cancer, especially breast cancer. In some embodiments, the dosage units contain at least 0.25 g, at least 0.27 g, at least 0.3 g, at least 0.35 g, or 0.35 g-4 g of a combination of Luteolin, Apigenin, Scutellarein, and Scutellarin. In some embodiments, the dosage units further comprise a package, such as a foil pack, a bottle, a sachet, a blister pack, or other sealed package. Thus, in some embodiments, the process of making the dosage unit includes a step of packaging the dosage unit in a package.

Illustrative amounts of active soluble matter (Luteolin, Apigenin, Scutellarein, and Scutellarin) in each dosage unit, or each dose, according to the present invention, are set forth in the following Table 2.

TABLE 2
Amounts of Active Soluble Matter (Luteolin, Apigenin, Scutellarein,
and Scutellarin) in Some Contemplated Dosages/Dosage Units
Described Herein

| Luteolin | Apigenin | Scutellarein | Scutellarin | Total |
|---|---|---|---|---|
| (mg/dose)* | (mg/dose)* | (mg/dose)* | (mg/dose)* | (mg/dose)** |
| 7 | 7 | 32 | 226 | 272 |
| 9 | 10 | 43 | 301 | 363 |
| 10 | 11 | 48 | 339 | 408 |
| 11 | 12 | 54 | 377 | 454 |
| 12 | 13 | 59 | 414 | 499 |
| 13 | 15 | 64 | 452 | 544 |
| 14 | 16 | 70 | 490 | 590 |
| 16 | 17 | 75 | 527 | 635 |
| 17 | 18 | 81 | 565 | 681 |
| 18 | 20 | 86 | 603 | 726 |
| 19 | 21 | 91 | 640 | 771 |
| 20 | 22 | 97 | 678 | 817 |
| 21 | 23 | 102 | 716 | 862 |
| 22 | 24 | 107 | 753 | 907 |
| 23 | 26 | 113 | 791 | 953 |
| 24 | 27 | 118 | 829 | 998 |
| 26 | 28 | 124 | 866 | 1044 |
| 27 | 29 | 129 | 904 | 1089 |
| 28 | 30 | 134 | 942 | 1134 |
| 29 | 32 | 140 | 979 | 1180 |
| 30 | 33 | 145 | 1017 | 1225 |
| 31 | 34 | 150 | 1055 | 1270 |
| 32 | 35 | 156 | 1093 | 1316 |
| 33 | 37 | 161 | 1130 | 1361 |
| 34 | 38 | 167 | 1168 | 1407 |
| 35 | 39 | 172 | 1206 | 1452 |
| 38 | 41 | 183 | 1281 | 1543 |
| 40 | 44 | 193 | 1356 | 1633 |
| 42 | 46 | 204 | 1432 | 1724 |
| 44 | 49 | 215 | 1507 | 1815 |
| 89 | 98 | 430 | 3014 | 3630 |

*±1 mg/dose;
**mg/dose ± 2 mg/dose

### Methods of Use

The pharmaceutical compositions and dosage units described herein may be used to treat cancer, especially breast and gynecological cancers. The inventor has conducted clinical trials in humans of compositions according to the invention and found that administration of 20 grams per day, 30 grams per day or 40 grams per day of soluble material extracted from Scutellaria barbata D. Don were well-tolerated and demonstrated efficacy against breast cancer, especially breast cancer with advanced breast cancer who had previously received at least one round of cancer therapy, an at least one round of chemotherapy. As treatment-refractory cancers of the breast are particularly difficult to treat, the inventor has provided a method of treating cancer in humans. In particular, the inventor has provided a method of treating breast cancer in humans, in addition to providing a method of treating one or more sub-types of cancers including metastatic breast cancers. Other cancers that may be treated include those that do not express estrogen receptors (ER-negative breast cancer) those that do not express progesterone (PR-negative breast cancers), those that do not express human epidermal growth hormone receptor 2 (HER2-negative breast cancers). It is noted in this regard that these categories of breast cancer are not mutually exclusive. For example, a breast cancer may be ER-negative and PR-negative (so-called double-negative breast cancer) or may be ER-negative, PR-negative and HER2-negative (triple-negative breast cancer). A triple negative breast cancer may be advanced and/or metastatic. A metastatic breast cancer may be, and often will be, one that has proven refractory to one or more previous therapeutic approaches. Thus, as used herein, the recitation of one characteristic of breast cancer (e.g. ER-negative) is not intended to exclude other characteristics (e.g. PR-negative) unless clearly stated.

Thus, some embodiments of the invention provide a method of treating cancer, comprising administering to a cancer patient an effective amount of a pharmaceutical composition comprising at least one excipient other than water, and at least one member of the group consisting of Luteolin, Apigenin, Scutellarein, and Scutellarin. In some embodiments, the composition comprises each of Apigenin, Luteolin, Scutellarein, Scutellarin, wherein at least one excipient other than water is selected from taste masking agents and sweeteners. In some embodiments, the composition is substantially free of high molecular weight compounds. In some embodiments, the cancer is breast cancer or a gynecological cancer. In some embodiments, the cancer is a breast cancer that is at least one of the following: advanced breast cancer, metastatic breast cancer, ER-negative breast cancer, PR-negative breast cancer, HER2-negative breast cancer, ER-negative and PR-negative breast cancer, ER-negative, PR-negative and HER2-negative breast cancer, or breast cancer that has not responded to at least one previous course of cancer treatment.

Some embodiments of the invention further provide a method of treating a cancer, e.g. a breast or gynecological cancer, by administering to a patient suffering from the cancer a pharmaceutical composition comprising at least about 0.25 g, at least about 0.27 g, at least about 0.3 g, at least about 0.35 g, or about 0.35 g to about 4 g of a combination of Luteolin, Apigenin, Scutellarein, and Scutellarin. In some embodiments, the method comprises administering to a patient a daily dose of about 0.25 g, about 0.27 g, about 0.3 g, about 0.35 g, about 0.4 g, about 0.45 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, of about 4 g of a combination of Luteolin, Apigenin, Scutellarein, and Scutellarin. In some embodiments, the cancer is breast cancer or a gynecological cancer. In some embodiments, the cancer is a breast cancer that is at least one of the following: advanced breast cancer, metastatic breast cancer, ER-negative breast cancer, PR-negative breast cancer, HER2-negative breast cancer, ER-negative and PR-negative breast cancer, ER-negative, PR-negative and HER2-negative breast cancer, or breast cancer that has not responded to at least one previous course of cancer treatment.

As mentioned above, the inventor has conducted clinical trials and has found that dosages exceeding 20 grams per day of soluble material extracted from Scutellaria barbata D. Don are well-tolerated and effective in a particularly hard-to-treat group of cancer patients. In addition, the inventor has found that the active compounds in the soluble material of an extract of Scutellaria barbata D. Don are one or more of Apigenin, Luteolin, Scutellarein and Scutellarin (preferably all four). Thus, some embodiments provide a method of treating cancer comprising administering to the patient a pharmaceutical dosage unit comprising at least 20 grams of an active pharmaceutical ingredient that contains at least one member of the group consisting of Apigenin, Luteolin, Scutellarein and Scutellarin. In some embodiments, the active pharmaceutical ingredient contains each of Apigenin, Luteolin, Scutellarein, and Scutellarin. In some embodiments, the dosage unit is an oral dosage unit. In some embodiments, the dosage unit further comprises at least one excipient other than water. In some embodiments, the dosage unit comprises at least one excipient selected from taste masking agents and sweeteners. In some embodiments, the method comprises administering to a patient a daily dose of soluble matter extracted from Scutellaria barbata D. Don that comprises at least about 0.25 g, about 0.27 g, about 0.3 g, about 0.35 g, about 0.4 g, about 0.45 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, of about 4 g of a combination of Luteolin, Apigenin, Scutellarein, and Scutellarin. In some embodiments, the cancer is a breast cancer that is at least one of the following: advanced breast cancer, metastatic breast cancer, ER-negative breast cancer, PR-negative breast cancer, HER2-negative breast cancer, ER-negative and PR-negative breast cancer, ER-negative, PR-negative and HER2-negative breast cancer, or breast cancer that has not responded to at least one previous course of cancer treatment.

Some embodiments described herein provide a method of treating cancer comprising administering to the patient at least 20 grams per day of an active pharmaceutical ingredient that contains at least one member of the group consisting of Apigenin, Luteolin, Scutellarein and Scutellarin. In some embodiments, the active pharmaceutical ingredient is administered in one to four doses per day. In some embodiments, the cancer is breast cancer or a gynecological cancer. In some embodiments, the cancer is a breast cancer that is at least one of the following: advanced breast cancer, metastatic breast cancer, ER-negative breast cancer, PR-negative breast cancer, HER2-negative breast cancer, ER-negative and PR-negative breast cancer, ER-negative, PR-negative and HER2-negative breast cancer, or breast cancer that has not responded to at least one previous course of cancer treatment. In some embodiments, the method comprises administering to a patient a daily dose of soluble matter extracted from Scutellaria barbata D. Don that comprises at least about 0.25 g, about 0.27 g, about 0.3 g, about 0.35 g, about 0.4 g, about 0.45 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, of about 4 g of a combination of Luteolin, Apigenin, Scutellarein, and Scutellarin.

Some embodiments, described herein provide a method of treating cancer comprising administering to the patient a pharmaceutical dosage unit comprising an active pharmaceutical ingredient containing at least 20 g of soluble material extracted from Scutellaria barbata D. Don. In some embodiments, the dosage unit is an oral dosage unit. In some embodiments, the dosage unit further comprises at least one excipient other than water. In some embodiments, the dosage unit comprises at least one excipient selected from taste masking agents and sweeteners. In some embodiments, the dosage unit comprises at least about 20 grams of the soluble material extracted from Scutellaria barbata D. Don.

Some embodiments further provide a method of treating cancer comprising daily administering to the patient an active pharmaceutical ingredient that contains at least 15 grams of soluble material extracted from Scutellaria barbata D. Don. In some embodiments, the active pharmaceutical ingredient is administered in one to four doses per day. In some embodiments, the cancer is breast cancer or a gynecological cancer. In some embodiments, the cancer is a breast cancer that is at least one of the following: advanced breast cancer, metastatic breast cancer, ER-negative breast cancer, PR-negative breast cancer, HER2-negative breast cancer, ER-negative and PR-negative breast cancer, ER-negative, PR-negative and HER2-negative breast cancer, or breast cancer that has not responded to at least one previous course of cancer treatment.

The inventor has found that in some embodiments, the particular dosage used to treat the patient is critical to a successful clinical outcome. Accordingly, in some embodiments the patient must be administered at least 20 g/day of soluble material extracted from Scutellaria barbata D. Don. In some embodiments, the dosage unit comprises at least about 20 grams of the active pharmaceutical ingredient. In some embodiments, the dosage unit comprises at about 20 grams to about 200 grams, about 20 grams to about 100 grams, about 20 grams to about 60 grams, about 20 grams, about 30 grams, about 40 grams, about 50 grams, about 60 grams, about 70 grams, about 80 grams, about 90 grams or about 100 grams of the active pharmaceutical ingredient. A preferred mode of administration is oral administration, preferably where the soluble material extracted from Scutellaria barbata D. Don is combined with at least one excipient other than water, such as a taste-masking agent, a sweetener or both.

Activity of an Extract of Scutellaria barbata D. Don In Vitro

Table 3A shows the degree of inhibition of the activity of several in vitro solid breast cancer tumor cell lines by the extract of this invention.

TABLE 3A

| MCF7 | SKBR3 | MDA-MB231 | BT474 | MCNeuA |
|---|---|---|---|---|
| ++ | ++ | ++ | + | ++ |

Table 3B shows the degree of inhibition of the activity of several in vitro solid cancer tumor cell lines by the extract of this invention.

TABLE 3B

| Lung | | Pancreatic | | Prostate | | Breast | | |
|---|---|---|---|---|---|---|---|---|
| Cancer | | Cancer | | Cancer | | Cancer | | Breast Normal |
| A549 | LLC | Panc1 | Panc02 | PC-3 | LNCaP | MCF7 | MCNeuA | HuMEC |
| + | ++ | + | ++ | + | + | ++ | ++ | − |
| 1424 | 492 | 1054 | 594 | 1035 | 1516 | 818 | 619 | |

− <50% inhibition,
+ 51-75% inhibition,
++ >75% inhibition,
IC₅₀ values (μg/ml)

It is an aspect of the present invention to isolate and characterize the active compounds in an extract from Scutellaria barbata D. Don (“BZL”). The extract loses activity when reconstituted after drying, as well as when the extract is separated through physical and chemical means.

As used herein, the terms “treat”, “treating” and “treatment” refer ameliorating one or more symptoms of a disease state. Successful treatment may be judged by attainment of stable disease, partial or total remission, or partial or total retardation of disease progression. One suitable end point for successful treatment is extension of life expectancy.

As used herein, “administer”, “administering” or “administration” refers to the delivery of an extract or extracts of this invention or of a pharmaceutical composition containing an extract or extracts of this invention to a patient in a manner suitable for the treatment of particular cancer being addressed.

A “patient” refers to a mammal having a tumor, especially a human, and more particularly a female human suffering from one or more gynecological cancers or breast cancer.

As used herein, the terms “effective amount” and “therapeutically effective amount” refer synonymously to that amount of a composition or dosage unit which in a patient population has the effect of (1) reducing the size of the tumor; (2) inhibiting (that is, slowing to some extent, preferably stopping) tumor metastasis; (3) inhibiting to some extent (that is slowing to some extent, preferably stopping) tumor growth; and/or; (4) relieving to some extent (or preferably eliminating) one or more symptoms associated with cancer; (5) stabilizing the growth of the tumor; (6) extending the time to disease progression; (7) improving overall survival.

As used herein, a “pharmaceutical composition” refers to a mixture of one or more of the compounds or combinations described herein with other chemical components, such as physiologically acceptable carriers and excipients. The purpose of a pharmacological composition is to facilitate administration of an extract or extracts of this invention to patient.

As used herein, the term “pharmaceutically acceptable” means that the agent or excipient is generally regarded as acceptable for use in a pharmaceutical composition.

As used herein, a “physiologically acceptable carrier” refers to a carrier or diluent that does not cause significant irritation to an organism and does not abrogate the biological activity and properties of the administered composition. Exemplary pharmaceutically acceptable carriers include solid and liquid diluents. Water, ethanol, propylene glycol, and glycerol are illustrative pharmaceutically acceptable liquid diluents; of these, water is preferred in some embodiments.

As used herein, an “excipient” refers to a pharmaceutically inert substance added to a pharmaceutical composition to further facilitate administration of a pharmaceutical composition of this invention. Examples, without limitation, of excipients include calcium carbonate, calcium phosphate, various sugars and types of starch, cellulose derivatives, gelatin, vegetable oils and polyethylene glycols. The groups of excipients and active pharmaceutical ingredients are considered mutually exclusive in the pharmaceutical arts. In some preferred embodiments, the excipient is a taste-masking agent, a sweetener, or both.

The term “excipient other than water” means that the excipient is or contains some excipient other than water, such as a taste-masking agent or a sweetener. Thus, the term “excipient other than water” would include an excipient that contained water and a sweetener or water and a taste-masking agent, but would exclude an excipient that contained water only. A pharmaceutical composition comprising an excipient other than water and an active pharmaceutical ingredient, for example, may contain the pharmaceutically active ingredient, water, and some other excipient, such as a taste masking agent and/or a sweetener.

As used herein, the terms “comprising”, “comprises”, “comprise” and grammatical variants thereof are inclusive or open-ended and do not exclude additional, unrecited elements or method steps. The terms “include”, “includes”, “contain”, “contains”, “containing” and grammatical variants thereof are likewise inclusive.

As used herein, the phrase “consisting of” excludes any element, step, or ingredient not specified in the following portion of the sentence.

As used herein, the phrase “consisting essentially of” limits the scope of the following part of the sentence to the specified materials or steps and those that do not materially affect the basic and novel characteristic(s) of the claimed invention.

As used herein, “BZL” is synonymous with “Scutellaria barbata D. Don.” The term “BZL101” refers to a specific extract of BZL, which has demonstrated activity against cancer cells. In particular, the aerial portions of Scutellaria barbata D. Don are intended.

### EXAMPLES

The herb from which the extracts of this invention were obtained were purchased from Shen Nong Herbs, Berkeley, Calif. Their identity was confirmed by reference to traditional pharmaceutical literature.

### Preparative Example 1

### Isolation of Active Compounds from Scutellaria barbata (BZL)

Dried Scutellaria barbata was extracted with 8:2 MeOH—H2O for 6 h and 12 h. The combined extracts were filtered, concentrated in vacuo, and sequentially partitioned with hexane and ethyl acetate (equal volume, repeated once). The combined ethyl acetate partitions were concentrated in vacuo and chromatographed over Sephadex lipophilic LH-20 media (˜160 g, 1800×25 mm i.d. column) under gravity flow using isocratic 9:0.5:0.5 MeOH-acetone-H2O or 100% MeOH. Fractions (40 ml) were collected and combined based on analytical HPLC (Table 3) and/or RF-TLC (1:1 10 mM ammonium acetate-MeCN) analysis.

Scutellarein (1), Isoscutellarein (2), Luteolin (3), and Apigenin (4) eluted in partially overlapping fractions from the Sephadex LH-20 column. Preparative HPLC method A (Table 3) was utilized to purify the individual compounds.

The flavanones Carthamidin (5) and Isocarthamidin (6) eluted together from the Sephadex LH-20 column in different fractions from the above flavones. Preparative HPLC method B (Table 3) was used to purify Carthamidin (5) and Isocarthamidin (6).

Isoscutellarein was identified based on LC/MS and 1D and 2D NMR analyses. All other compounds (1, 3-6) were identified based on LC/MS and NMR comparison with a commercial reference standard or from an authenticated standard from synthesis. NMR spectra were recorded using a Varian Mercury Plus 400 MHz. The HPLC and UV spectrum were recorded using an Agilent Technologies 1200 Series HPLC system, equipped with a DAD detector, and using a Phenomenex Luna C18 (150×2.1 mm, 3 μm) column. The molecular mass was determined using an Applied Agilent Technologies 6210 TOF LC/MS in the negative mode. A summary of the properties of the instrumentation used is set forth in Table 1-1.

TABLE 1-1

| | Column | | Flow rate |
|---|---|---|---|
| Method | (l × .i.d.; particle size) | Column description | (ml/min) |
| Analytical | 150 × 4.6 mm; 5 μm | Phenomenex | 1 |
| | | Luna C18(2) | |
| Preparative A | 150 × 21.1 mm; 5 μm | Phenomenex | 20 |
| | | Luna C18(2) | |
| Preparative B | 150 × 50.0 mm; 5 μm | Waters Sunfire, C18 | 120 |

[Table 1-1: HPLC methods.] The columns listed below were used in isolating compounds 1-6. The same solvent gradient was used for chromatography for all HPLC runs, only the flow rate was different as specified. Gradient: solvent A: 0.1% TFA. solvent B: MeCN; Linear gradient from 10% B to 60% B in 30 min with no upfront hold.

The NMR data used to identify compounds 1-6 are set forth below.

Scutellarein (1): CAS#529-53-3; LC/MS [M-H]− m/z 285.0425. Formula 1 shows the key HMBC correlations of compound (1). The NMR data for compound 1 are set forth in Table 1-2.

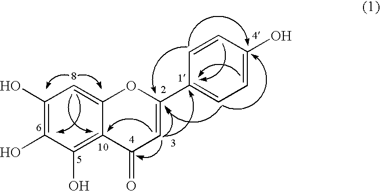

TABLE 1-2
¹H (pyridine-d₅, 400 MHz, mult, int, J in Hz) and ¹³C
(pyridine-d₅, 100 MHz) NMR data for compound 1

| Position | δ_{C} | δ_{H} | δ_{C}* | δ_{H}* |
|---|---|---|---|---|
| 1 | | | | |
| 2 | 164.9, s | | 163.6, s | |
| 3 | 103.8, d | 6.93 (1H, s) | 102.4, d | 6.73 (1H, s) |
| 4 | 183.6, s | | 182.1, s | |
| 5 | 148.9, s | | 147.1, s | |
| 6 | 151.6, s | | 129.2, s | |
| 7 | 155.9, s | | 153.4, s | |
| 8 | 95.6, d | 7.06 (1H, s) | 93.9, d | 6.56 (1H, s) |
| 9 | 131.7, s | | 149.7, s | |
| 10 | 105.8, s | | 104.1, s | |
| 1′ | 123.2, s | | 121.6, s | |
| 2′ and 6′ | 129.3, d | 7.95 (2H, d, 8.8) | 128.4, d | 7.90 (2H, d, 8.6) |
| 3″ and 5″ | 117.3, d | 7.24 (2H, d, 8.8) | 116.0, d | 6.91 (2H, d, 8.6) |
| 4″ | 163.0, s | | 161.1, s | |

*Data from HongJun Xia, Feng Qiu, Shan Zhu, TieYing Zhang, GeXia Qu, and XinSheng Yao, 2007. Isolation and identification of ten metabolites of breviscapine in rat urine. Biological Pharmaceutical Bulletin, 30 (7): 1308-1316., which were recorded in DMSO-d₆.

Isoscutellarein (2): CAS#41440-05-5; LC/MS [M-H]− m/z 285. Formula (2) shows the key HMBC correlations of compound (2). NMR data for compound 2 are set forth in Table 1-3.

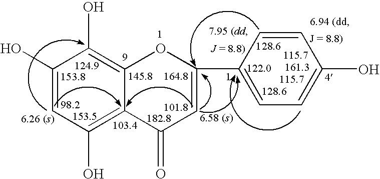

TABLE 1-3
¹H (methanol-d₄, 400 MHz, mult, int, J in Hz) and ¹³C
(methanol-d₄, 100 MHz) NMR data for compound 2

| Position | δ_{C} | δ_{H} |
|---|---|---|
| 1 | | |
| 2 | 164.8 | |
| 3 | 101.8 | 6.58 (s) |
| 4 | 182.8 | |
| 5 | 153.5 | |
| 6 | 98.2 | 6.26 (s) |
| 7 | 153.8 | |
| 8 | 124.9 | |
| 9 | 145.8 | |
| 10 | 103.4 | |
| 1′ | 122.0 | |
| 2′ | 128.6 | 7.95 (dd, J = 8.8) |
| 3′ | 115.7 | 6.94 (dd, J = 8.8) |
| 4′ | 161.3 | |

Luteolin (3): CAS#491-70-3; LC/MS [M-H]− m/z 285.0403. Formula 3 shows the key HMBC correlations of compound (3). NMR data for compound 3 are set forth in Table 1-4.

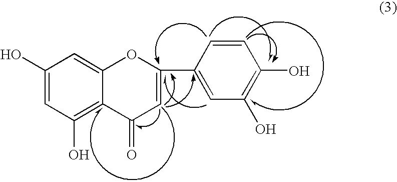

TABLE 1-4
¹H (acetone-d6, 400 MHz, mult, int, J in Hz) and ¹³C
(acetone-d, 100 MHz) NMR data for compound 3

| Position | δ_{C} | δ_{H} |
|---|---|---|
| 1 | | |
| 2 | 164.5 | |
| 3 | 103.4 | 6.58 (s) |
| 4 | 182.4 | |
| 5 | 162.3 | |
| 6 | 98.9 | 6.24 (d, J = 2.0) |
| 7 | 164.3 | |
| 8 | 94.0 | 6.51 (d, J = 2.0) |
| 9 | 158.2 | |
| 10 | 104.5 | |
| 1′ | 122.9 | 7.47 (d, J = 2.4) |
| 2′ | 113.3 | |
| 3′ | 145.8 | |
| 4′ | 149.5 | |
| 5′ | 115.8 | 6.98 (dd, J = 8.8, 2.4) |
| 6′ | 119.3 | 7.46 (dd, J = 8.8, 2.4) |

Apigenin (4): CAS#520-36-5; LC/MS [M-H]− m/z 269.04479. Formula (4) shows the structure of Apigenin (4).

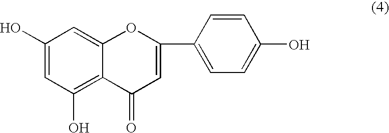

Carthamidin (5): CAS#479-54-9; LC/MS [M-H]− m/z 287. Formula (5) shows the key HMBC correlations of compound (5). NMR Data for compound 5 are set forth in Table 1-5.

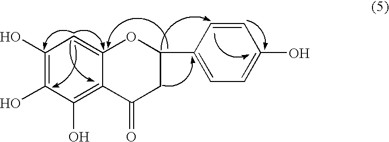

TABLE 1-5
¹H (methanol-d₄, 400 MHz, mult, int, J in Hz) and ¹³C
(methanol-d₄, 100 MHz) NMR data for compound 5

| Position | δ_{C} | δ_{H} |
|---|---|---|
| 1 | | 5.28 (dd, J = 2.8, 20.8) |
| 2 | 79.2 | 2.67 (dd, J = 4.4, 12.8) |
| 3 | 42.8 | 3.08 (dd, J = 2.8, 14.0 |
| 4 | 197.1 | |
| 5 | 149.7 | |
| 6 | 126.1 | |
| 7 | 155.2 | |
| 8 | 94.4 | 5.95 (s) |
| 9 | 156 | |
| 10 | 101.8 | |
| 1′ | 129.1 | |
| 2′ | 127.5 | 7.31 (d, J = 8.0) |
| 3′ | 114.8 | 6.81 (d, J = 8.0) |
| 4′ | 157.8 | |
| 5′ | 114.8 | 6.81 (d, J = 8.0) |
| 6′ | 127.5 | 7.31 (d, J = 8.0) |

Isocarthamidin (6): CAS#2569-76-8; LC/MS [M-H]− m/z 287. Formula (6) shows the key HMBC correlations of compound (6). NMR data for compound 6 are set forth in Table 1-6.

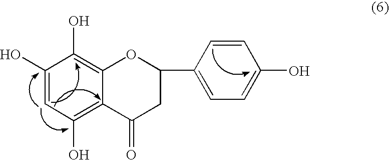

TABLE 1-6
¹H (methanol-d₄, 400 MHz, mult, int, J in Hz) and ¹³C
(methanol-d₄, 100 MHz) NMR data for compound 6

| Position | δ_{C} | δ_{H} |
|---|---|---|
| 1 | | |
| 2 | 79.4 | 5.38 (dd, J = 2.8, 9.2) |
| 3 | 42.7 | 2.73 (dd, J = 4.8, 12.4) 3.74 (dd, J = 3.2, 14.0 |
| 4 | 196.5 | |
| 5 | 149 | |
| 6 | 95.2 | 5.94 (s) |
| 7 | 156.6 | |
| 8 | 125.3 | |
| 9 | 156.1 | |
| 10 | 101.7 | |
| 1′ | 129.6 | |
| 2′ | 127.8 | 7.37 (d, J = 8.8) |
| 3′ | 114.8 | 6.8 (d, J = 8.8 |
| 4′ | 157.6 | |
| 5′ | 114.8 | |
| 6′ | 127.8 | 7.37 (d, J = 8.8) |

### Preparative Example 2

### Preparation of BZL101 for Human In Vivo Experiments

BZL101 is an aqueous extract of the aerial part of Scutellaria Barbata D. Don of the Lamiaceae family. Herba Scutellaria Barbata D. Don (Chinese pin yin transliteration—Ban Zhi Lian (BZL)) is grown mainly in areas southeastern of the Yellow River (Huang Po) in the provinces of Sichuan, Jiangsu, Jiangxi, Fujian, Guangdong, Guangxi and Shaanxi. The plant is harvested in late summer and early autumn after it blooms. The aerial part (leaves and stems) is cut from the root and is used as starting material (BZL). The aerial part of the herb is dried in the sun, packed as a whole plant. The herb is identified and verified through botanical, morphological and chemical characteristics to ensure purity.

A single dose of BZL101 is made through the following procedure and is termed BZL101 (Bionovo, Inc., Emeryville, Calif.).
180 grams of the raw herb is ground to fine powder (25 mesh)
The powder is mixed with 1800 ml of distilled water to form a slurry
The slurry is than simmered at 70-72° C. for 60 minutes
The extract is decanted and filtered through 22 μm filter
The supernatant weight after extraction is 168 gm
The volume of the solution is 1750 ml
The extract is concentrated with a vacuum evaporator to reduce the volume of water to 350 ml which constitutes a 5:1 concentration of the original solution
The dry weight of soluble material in the extract is 12 gm
It is packaged in a sterile, vacuum sealed container
Testing for bacteria, yeast and heavy metals are preformed by an accredited laboratory

For higher doses (e.g. 20, 30 and 40 grams per day) the quantities of raw herb (aerial parts of Scutellaria barbata D. Don and water are scaled proportionately, with proportionate resulting amount of dry weight of soluble material.

### Example 1

### Characterization of Actives from Scutellaria Barbata D. Don

Rationale

BZL101 induces cell death in breast cancer cells but not in non-transformed mammary epithelial cells. This selective cytotoxicity is based on strong induction by BZL101 of reactive oxygen species (ROS) in tumor cells. As a consequence, BZL101-treated cancer cells develop extensive oxidative DNA damage and succumb to necrotic death. Data from the expression profiling of cells treated with BZL101 are strongly supportive of a death pathway that involves oxidative stress, DNA damage and activation of death-promoting genes. In breast cancer cells, oxidative damage induced by BZL101 leads to the hyperactivation of poly (ADP-ribose) polymerase (PARP), followed by a sustained decrease in levels of NAD and depletion of ATP, neither of which are observed in non-transformed cells. The hyperactivation of PARP is instrumental in the necrotic death program induced by BZL101, because inhibition of PARP results in suppression of necrosis and activation of the apoptotic death program. BZL101 treatment leads to the selective inhibition of glycolysis in tumor cells, which is evident from the decrease in the enzymatic activities within the glycolytic pathway and the inhibition of lactate production. Because tumor cells frequently rely on glycolysis for energy production, the observed inhibition of glycolysis is likely a key factor in the energetic collapse and necrotic death that occurs selectively in breast cancer cells. The promising selectivity of BZL101 towards cancer cells is based on metabolic differences between highly glycolytic tumor cells and normal cells.

Several types of experiments were conducted with individual compounds isolated from BZL101.

A total of seven purified compounds from BZL101 were tested for several biological activities present in the total aqueous BZL101 extract: induction of ROS, DNA damage and cell death. The following parameters were examined:

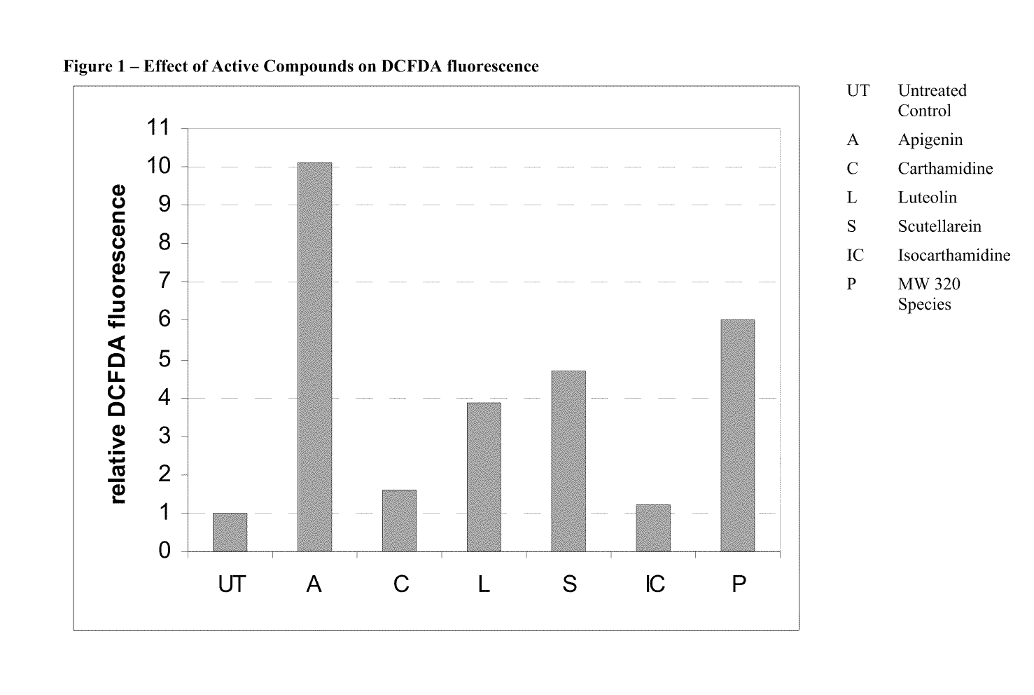

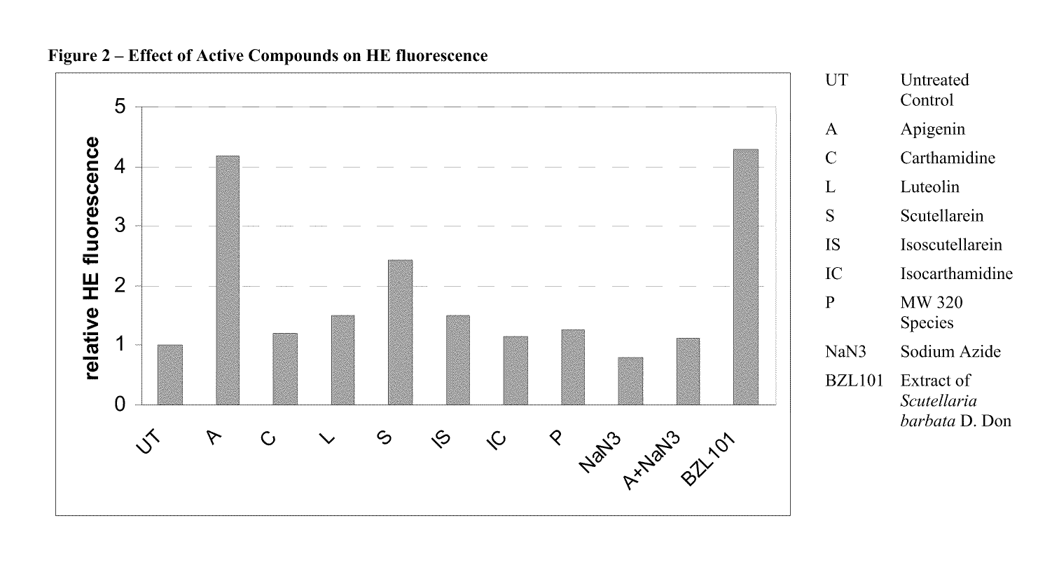

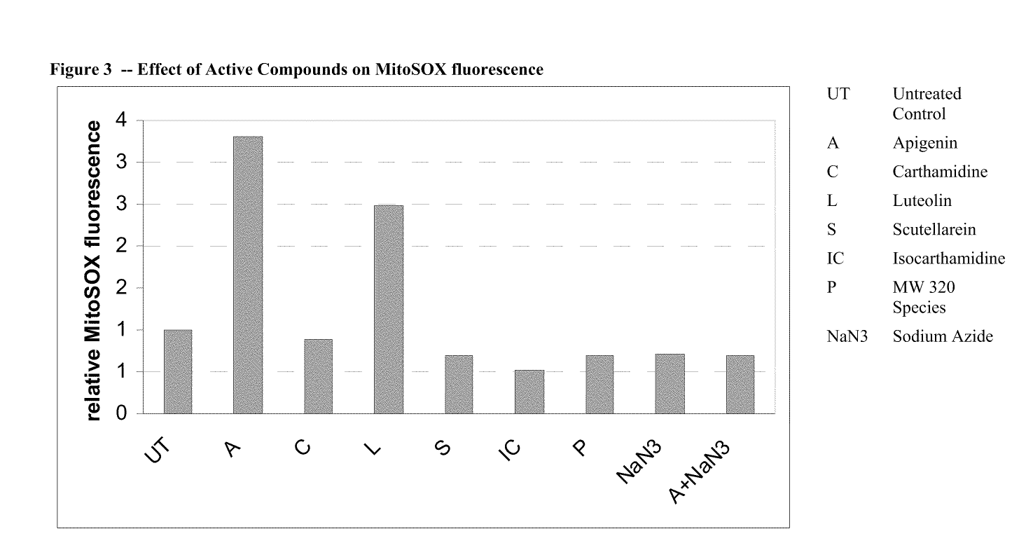

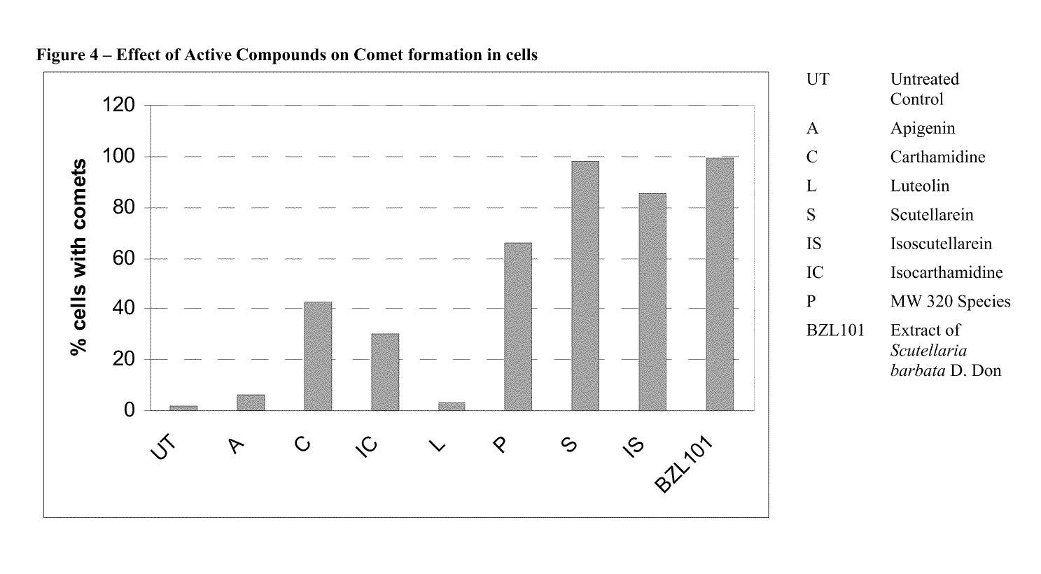

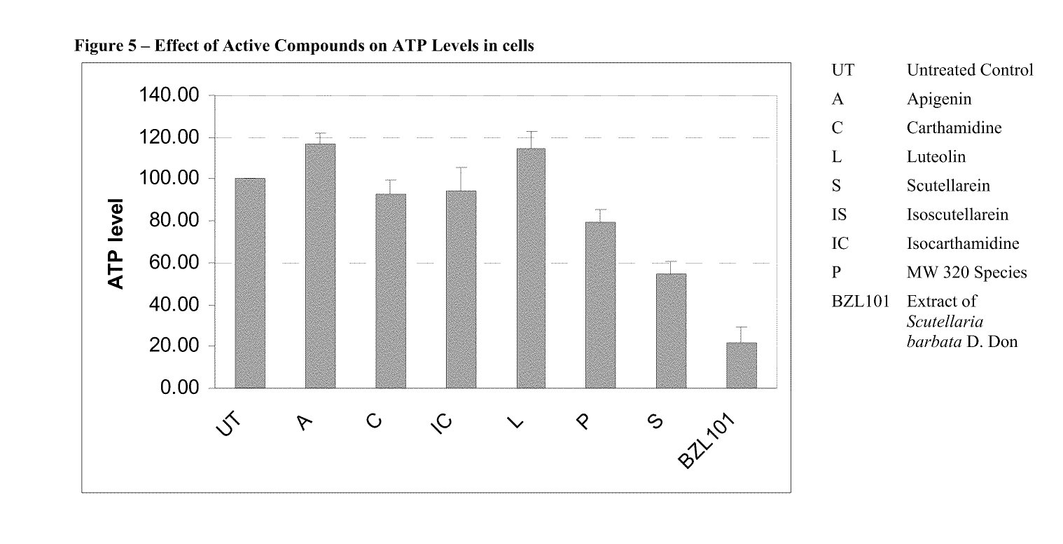

1. Induction of the loss of the mitochondrial transmembrane potential (MTP). All of compounds tested induced loss of MTP.
2. Induction of reactive oxygen species (ROS). Induced fluorescence from the cell permeable indicators of ROS such as dihydroethidium (specific for superoxide) (FIG. 2), CM-H2DCFDA (most types of ROS) (FIG. 1) and MitoSOX (mitochondrially derived superoxide) (FIG. 3) was studied using a fluorescent plate reader and FACS.
3. Compounds were also tested for the potential cellular sources of ROS induced using either specific indicators for ROS of mitochondrial origin, and/or specific inhibitors of ROS production by known sources such as mitochondria, ubiquinone oxidoreductase NQO (mitochondrial complex I) and NADPH oxidases.
4. Compounds were tested for induction of DNA damage using test known as comet assay that allows detection of DNA damage in individual cells. (FIG. 4)
5. The induction of death in cells treated with the compounds was examined using propidium iodide test for cell permeability followed by analysis on FACS.
6. The mode of cell death (i.e., apoptosis versus necrosis) was studied using several criteria: conversion of cells to positivity for binding Annexin V; DNA fragmentation (characteristic of apoptotic death) and decrease in cellular ATP levels (commonly observed during necrotic death) (FIG. 5). 1. Induction of the loss of the mitochondrial transmembrane potential (MTP). All of compounds tested induced loss of MTP.
2. Induction of reactive oxygen species (ROS). Induced fluorescence from the cell permeable indicators of ROS such as dihydroethidium (specific for superoxide) (FIG. 2), CM-H2DCFDA (most types of ROS) (FIG. 1) and MitoSOX (mitochondrially derived superoxide) (FIG. 3) was studied using a fluorescent plate reader and FACS.
3. Compounds were also tested for the potential cellular sources of ROS induced using either specific indicators for ROS of mitochondrial origin, and/or specific inhibitors of ROS production by known sources such as mitochondria, ubiquinone oxidoreductase NQO (mitochondrial complex I) and NADPH oxidases.
4. Compounds were tested for induction of DNA damage using test known as comet assay that allows detection of DNA damage in individual cells. (FIG. 4)
5. The induction of death in cells treated with the compounds was examined using propidium iodide test for cell permeability followed by analysis on FACS.
6. The mode of cell death (i.e., apoptosis versus necrosis) was studied using several criteria: conversion of cells to positivity for binding Annexin V; DNA fragmentation (characteristic of apoptotic death) and decrease in cellular ATP levels (commonly observed during necrotic death) (FIG. 5).

The data depicted in FIGS. 1-5 are summarized in Table 1-7, below.

TABLE 1-7
Summary of the effects that compounds isolated from BZL101 have on breast
cancer cells

| | | | | | | Induction | |
|---|---|---|---|---|---|---|---|
| | Induction | | | | | of | |
| | of ROS | | | | | apoptosis | Depletion |
| | (peroxide, | | Induction of | | | (Annexin | of ATP |
| | superoxide, | | superoxide | Induction | Induction | V staining, | (indicative |
| | radicals, | Induction of | specifically | of DNA | of cells | DNA | of |
| | etc) | superoxide | in mitochondria | damage | death | fragmentation) | necrosis) |
| Apigenin | ++++ | ++++ | + | − | ++++ | ++++ | − |
| Luteolin | ++ | ++ | + | − | +++ | +++ | − |
| MW 320 Species | ++++ | + | − | ++ | ++ | − | + |
| Scutellarein | +++ | + | − | ++++ | ++ | − | + |
| Isoscutellarein | +++ | ND | ND | +++ | ++ | − | ND |
| Carthamidin | + | − | − | ++ | +/− | − | +/− |
| Isocarthamidin | + | − | − | ++ | +/− | − | +/− |

Two breast cancer cell lines, MDA MB 231 and SKBr3, were used in the experiments summarized in Table 1-7, with similar results.

Results

All of the tested compounds induced loss of the mitochondrial transmembrane potential, ranging from 25 to 90% loss of MTP compared to mock-treated cells (not shown).

Most of the compounds have cytotoxic activities; but the degree of cytotoxicity of each is different (Table 1-7). The compounds have differential effect on induction of reactive oxygen species (ROS), DNA damage and energy status of cells. (FIGS. 1-5). BZL101 extract thus contains a number of compounds with potentially different modes of cell death induction.

Analysis of the mechanism of death induction by different compounds reveals at least two different modes of cytotoxicity:

All compounds tested induce cellular ROS within minutes of treatment as determined by loading cells with using the oxidant-sensitive fluorescent probe 5-(and-6)-chloromethyl-2′,7′-dichlorodihydrofluorescein diacetate acetyl ester (CM-H₂DCFDA or DCFDA). DCFDA is nonfluorescent in reduced form and is readily membrane-permeant. Cellular esterases cleave its acetate groups. The thiol-reactive chloromethyl group then binds to cellular thiols, trapping the dye inside the cell, where oxidation converts it to the fluorescent form. CM-H₂ DCFDA is oxidized by cellular hydrogen peroxide, hydroxyl radicals, and various free radical products lying downstream from hydrogen peroxide. It is relatively insensitive to oxidation by superoxide. However, because hydrogen peroxide is produced by dismutation of superoxide, CM-H₂ DCFDA serves as an indirect indicator of superoxide production. As seen in FIG. 1, CM-H₂DCFDA is oxidized within cells by all tested BZL101 compounds, though levels of total ROS induced are different.

All the tested compounds also induced superoxide, as determined by staining of cells with dihydroethidium, a cell-permeant indicator that is oxidized selectively by superoxide. Cytosolic dihydroethidium exhibits blue fluorescence; however, once this probe is oxidized to ethidium by superoxide, it intercalates within the cell's DNA, staining its nucleus a bright fluorescent red, which is easily detected by flow cytometric methods (FIG. 2).

Two flavonoids, Apigenin and Luteolin, induce generation of superoxide whose origin is identified as mitochondrial. A specific detector of mitochondrially derived superoxide, MitoSOX, was converted to its fluorescent form by Apigenin and Luteolin, but not by other compounds. In addition, an inhibitor of mitochondrial respiration (sodium azide, NaN₃) and an inhibitor of mitochondrial complex I (dicumarol, not shown) prevented generation of superoxide by Apigenin and Luteolin (FIG. 3).

Apigenin and Luteolin are distinct from other compounds in that they do not induce DNA damage (FIG. 4). However, both are cytotoxic and induce significant cell death characterized as apoptotic based on: annexin V binding, DNA fragmentation, and slight but consistent increase in ATP levels observed during first hours of treatment (FIG. 5). All these features are hallmarks of apoptotic death.

Five compounds that are identified as either tetrahydroxyflavones (Scutellarein, Isoscutellarein, Carthamidin and Isocarthamidin) or pentahydroxylflavone (no name) induce cell death via a distinct mechanism. They induce ROS, but of extra-mitochondrial origin (the source remains to be determined). These compounds also induce DNA damage whose extent seems to correlate with the level of induction of ROS. It is possible that the type of ROS induced by Scutellarein and the like compounds is particularly active in inducing DNA damage, such as singlet oxygen. It is reasonable to assume that most ROS induces by these compounds are not superoxide, since superoxide is not membrane permeable and cannot induce direct oxidative DNA damage. However, superoxide can be quickly converted in cells to peroxide, which is can damage DNA directly.

Similarly to the total BZL101 extract, five compounds mentioned above induce a decrease in the levels of cellular ATP. Loss of ATP, along with lack or low staining for Annexin V, is more consistent with necrotic mode of cell death.

Figure Legends:

FIG. 1. Induction of ROS in SKBr3 cells as determined by staining with CM-H₂ DCFDA. The indicated compounds were added to cells at 20 μg/ml growth medium, followed by addition of 10 μM CM-H₂ DCFDA. Inhibitor of mitochondrial respiration, NaN3, was added at 10 mM. After 30 minute incubation, cells were washed in PBS and analyzed on FACScan for fluorescence. The compound names are abbreviated in this and other Figures as follows: A—Apigenin; C—Carthamidin; L—Luteolin; S—Scutellarein; IC—Isocarthamidin; IS—Isoscutellarein; P—a species having a molecular weight of 320 (believed to be a pentahydroxylflavone).

FIG. 2. Induction of superoxide in SKBr3 cells as determined by staining with dihydroethidium. The indicated compounds were added to cells followed by addition of 5 μM dihydroethidium. After 20 minute incubation, cells were washed in PBS and analyzed on FACScan for fluorescence.

FIG. 3. Cells were stained with MitoSOX indicator of mitochondrially derived superoxide. Treatments were as described in the Legends to FIG. 2.

FIG. 4. Induction of DNA damage in SKBr3 cells by compounds isolated from BZL101 was analyzed using comet assays. Cells were treated with the indicated compounds at 20 μg/ml for 15 minutes and analyzed for DNA damage using the Comet assay kit from Trevigen according to the manufacturer's instructions. Briefly, cells were harvested, washed and resuspended with PBS. The cells were combined with molten, low melting point agarose at 37° C. and pipetted unto Comet slides. The agarose was allowed to solidify at 4° C. for 30-40 min and immersed in cold lysis solution (Trevigen, Inc.) for 30 min at 4° C. The slides were immersed into freshly prepared alkali solution (300 mM NaCl and 1 mM EDTA) for 20 min and subjected to electrophoresis in the same alkaline buffer at 300 mA for 30-40 min. Slides were rinsed in water and then fixed in 70% ethanol for 5 min. After air-drying, the nuclei were stained with Sybr green (Trevigen, Inc.) and viewed under a fluorescence microscope. Percentages of cells with comets were quantified by an observer blinded to the identity of the slides.

FIG. 5. SKBr3 cells were plated on 96 well plates and treated with the indicated compounds for four hours. Cells were lysed in situ and ATP content was analyzed using the ATP Bioluminescence Assay Kit HSII from Roche, on a 96 well plate-based luminometer.

Conclusions:

BZL101 extract contains chemical compounds with cytotoxic activities. These compounds exhibit different effects on mitochondria and cellular DNA, but all have cytotoxic activity towards human cancer cells. Two of the identified compounds, Apigenin and Luteolin induce mitochondrial superoxide and apoptotic death that is executed through the mitochondrial, or intrinsic, pathway.

The other five compounds, in particular Scutellarein and Isoscutellarein, induce ROS followed by DNA damage and cell death that has hallmarks of programmed necrosis.

### Example 2

### Separation and Synergistic Activity of Actives Extracted from Scutellaria barbata D. Don

As demonstrated in Example 2, several compounds extracted from Scutellaria barbata D. Don were shown to induce generation of reactive oxygen species (ROS), DNA damage and cell death. In order to better understand the combined activities of the isolated species, several flavanones and flavones isolated from Scutellaria barbata were tested individually and in combination. The flavanones and flavones tested are depicted in FIG. 8. These compounds 1-8 from were tested for induction of ROS, DNA damage and cell death, as described in Example 2. The results of these tests are set forth in Tables 10 and 11, below.

TABLE 11
Scutellaria barbata extracted compounds are active - some are synergistic

| | Induction of ROS | | |
|---|---|---|---|
| | (fold) | DNA damage | Cell death |
| cmpd 1 | 0.9 | − | ND |
| cmpd 2 | 1.2 | − | − |
| cmpd 3 | 2.4 | + | ND |
| cmpd 4 | 0.6 | − | − |
| cmpd 5 (Scutellarein) | 2.4 | + | + |
| cmpd 6 (Isoscutellarein) | 1.5 | +/− | ++ |
| cmpd 7 (Luteolin) | 1.9 | + | ct+ |
| cmpd 9 (Apigenin) | 1.5 | ND | ++ |
| cmpd 8 pentaOH SBO8- | 0.8 | ND | − |
| 11-67) | | | |
| cmpd 7 + 6* | 2.6 | ND | ++++ |
| cmpd 7 + 9** | 2.9 | ND | ++++ |
| cmpd 6 + 9*** | 1.2 | ND | ++ |

*Total concentration of cmpd 7 + 6 equivalent to that of 7 alone or 6 alone
**Total concentration of cmpd 7 + 9 equivalent to that of 7 alone or 9 alone
***Total concentration of cmpd 6 + 9 equivalent to that of 6 alone or 9 alone

TABLE 12
Synergistic activity of compounds extracted from Scutellaria barbata

| | Induction of ROS | | |
|---|---|---|---|
| | (fold) | DNA damage | Cell death |
| cmpd 6 | 1.5 | +/− | ++ |
| cmpd 7 | 1.9 | + | + |
| cmpd 9 | 1.5 | − | ++ |
| cmpd 7 + 6* | 2.6 | ND | ++++ |
| cmpd 7 + 9** | 2.9 | ND | ++++ |
| cmpd 6 + 9*** | 1.2 | ND | ++ |

*Total concentration of cmpd 7 + 6 equivalent to that of 7 alone or 6 alone
**Total concentration of cmpd 7 + 9 equivalent to that of 7 alone or 9 alone
***Total concentration of cmpd 6 + 9 equivalent to that of 6 alone or 9 alone

As can be seen in tables 10 and 11, a combination of Luteolin and Isoscutellarein is far more effective at inducing generation of reactive oxygen species (ROS) and cell death than is the same concentration of Luteolin alone. Likewise, the combination of Luteolin and Isoscutellarein is far more effective at inducing generation of reactive oxygen species (ROS) and cell death than is the same concentration of Isoscutellarein alone. In this sense, the combination of Isoscutellarein and Luteolin is considered to have a synergistic effect on induction of ROS generation and cell death.

Also apparent from tables 10 and 11, is the fact that a combination of Luteolin and Apigenin is far more effective at inducing generation of reactive oxygen species (ROS) and cell death than is the same concentration of Luteolin alone. Likewise, the combination of Luteolin and Apigenin is far more effective at inducing generation of reactive oxygen species (ROS) and cell death than is the same concentration of Apigenin alone. In this sense, the combination of Apigenin and Luteolin is considered to have a synergistic effect on induction of ROS generation and cell death.

As can be seen in tables 10 and 11, a combination of Apigenin and Isoscutellarein is far more effective at inducing generation of reactive oxygen species (ROS) and cell death than is the same concentration of Apigenin alone. Likewise, the combination of Apigenin and Isoscutellarein is far more effective at inducing generation of reactive oxygen species (ROS) and cell death than is the same concentration of Isoscutellarein alone. In this sense, the combination of Isoscutellarein and Apigenin is considered to have a synergistic effect on induction of ROS generation and cell death.

The results set forth in Tables 10 and 11 were confirmed by performing the experiments in two different breast cancer cell lines.

### Example 4

### In Vivo Efficacy of Actives Derived from BZL101 in Humans

In order to demonstrate the safety and clinical activity of oral BZL101, a combination of active compounds isolated from Scutellaria Barbata D. Don is studied in human patients with advanced breast cancer.

Eligible patients have histologically confirmed metastatic breast cancer and measurable disease. Patients do not receive any other chemotherapy, hormone therapy or herbal medicine during the trial. Patients receive 350 ml (equivalent to 0.00001-1 gram each of one, two, three, four, five or all members of the group consisting of Apigenin, Luteolin, Scutellarein and Scutellarin) of drug per day until disease progression, toxicity or personal preference caused them to discontinue. The primary endpoints are safety, toxicity and tumor response.

Patients are enrolled and receive drug. Mean age and mean number of prior treatments are recorded. Hematologic, and grade III or IV non-hematologic, adverse events (AEs), if any, are tracked and recorded. Patients who report grade I and II adverse events, such as nausea, diarrhea, headache, flatulence, vomiting, constipation, and fatigue, if any, are noted and recorded. Patients who are evaluable for response are evaluated and those with stable disease (SD) for >90 days and those with SD for >180 days are noted and recorded. Patients who have minor objective tumor regression are also noted and recorded.

Patients are enrolled at one or more suitable research centers and sign informed consent approved by local institutional review boards. Patients are excluded from the study for the following: extensive liver involvement (>50% of liver parenchyma), lymphangitic pulmonary involvement, central nervous system involvement or spinal cord compression not stabilized by therapy for >3 months, a history of multiple or severe food or medicine allergies and organ or marrow dysfunction as defined by creatinine >2.0 g/dl, total bilirubin >1.7 g/dl, white blood cell count <2,500 cells/μL and platelet count <75,000 mm³. (“dl”=deciliter(s).)

Safety monitoring is conducted on a continuous basis and patients are seen by a physician for examination at baseline at every Y weeks. Adverse events are graded using Common Toxicity Criteria version 2, assigned a category by organ system and coded in relation to study drug as remote, possible, probably or definitely related. Baseline tumor assessments are done within 14 days of initiation of study drug and every three months. Responses are assessed using RECIST criteria. Study drug is administered at every visit, and at this visit compliance and a review of dosages taken was performed. Study drug is provided as a liquid in a sealed and labeled aluminum packet containing a full daily dose that is administered in a split dose twice a day. Daily study drug is administered until the determination of tumor progression or dose limiting toxicity is encountered, or until the subject decides to voluntarily discontinue, in which case, the reason for discontinuation is obtained.

Results

Results of the above study are noted and evaluated based upon meeting the study endpoints.

### Example 4

### Active Concentrations in Soluble Matter Extracted from Scutellaria barbata D. Don

BZL101 is prepared as described herein. Active compounds, Luteolin, Apigenin, Scutellarein, and Scutellarin, are identified and quantified relative to 1 mg of BZL101. The mass of each of Luteolin, Apigenin, Scutellarein, and Scutellarin in 1 mg of soluble matter in BZL101 is given in table 4-1:

TABLE 4-1
Proportions of Luteolin, Apigenin, Scutellarein, and Scutellarin
per mg of BZL101

| | Luteolin | Apigenin | Scutellarein | Scutellarin | Total |
|---|---|---|---|---|---|
| Proportion | 0.4435 | 0.4875 | 2.1496 | 15.069 | 18.1496 |
| (mcg/mg) | | | | | |
| SD | 0.0465 | 0.0435 | 0.2375 | 2.0547 | 5.078603 |
| (±mcg/mg) | | | | | |

As can be seen in Table 4-1, in this illustrative and non-limiting example, 1 mg of soluble matter extracted from Scutellaria barbata D. Don contains about 0.44 μg±0.05 μg Luteolin, 0.49 μg±0.04 μg Apigenin, 2.1 μg-0.2 μg Scutellarein and 15 μg-2 μg of Scutellarin. Thus each mg of dry soluble matter extracted from Scutellaria barbata D. Don contains about 18 μg-5 μg of the combination of Luteolin, Apigenin, Scutellarein, and Scutellarin in proportions of about 1:1.1:4.8:34.

### Example 5

### Scutellaria barbata D. Don Extract in the Treatment of Treatment-Refractive Metastatic Breast Cancer

Treatment of metastatic breast cancer patients was conducted with an extract of Scutellaria barbata D. Don (“BZL101”). The extract, BZL101, was prepared essentially as described hereinabove, and was given to patients who had undergone one or more courses of treatment for metastatic breast cancer. BZL101 was given either once per day (q.d.) or twice per day (b.i.d.) as described below. 20 gram, 30 gram and 40 gram doses proved to be well tolerated, despite their being far higher than ever reported in the literature relating to Scutellaria barbata. Additionally, several patients demonstrated efficacy as discussed below.

BZL101 is an extract of Scutellaria barbata, which evinces a novel mechanism of action. Normal cells depend on citric acid cycle (>85%) and glycolysis (<7%) for energy production. Cancer cells depend on glycolysis (>85%) for energy production. BZL101 inhibits energy production by inhibiting glycolysis. BZL101 causes DNA damage and cancer cell death. BZL101 does NOT cause cell death in normal cells.

The following bases have been propounded for the selective cytotoxic activity of BZL101 in cancer cells: Tumor cells rely on glycolysis for energy production. This is associated with increased endogenous levels of reactive oxygen species (ROS). Normal cells rely on oxidative phosphorylation for their energy needs. BZL101 treatment further increases ROS levels in tumor cells leading to hyper-activation of poly ADP ribose polymerase (PARP) and massive oxidative DNA damage. In normal cells BZL101 treatment results only in mild increase of ROS levels and moderate DNA damage without PARP activation.

Activation of PARP depletes NAD+/NADH (substrate for synthesis of poly ADP-ribose) and ATP stores. Glycolysis uses cytosolic NAD+ as a substrate to generate ATP and is inhibited by lack of NAD+. (Oxidative phosphorylation uses mitochondrial NAD+ to generate ATP and is generally not affected by PARP activation). Depletion of NAD+ and ATP by BZL101-induced PARP activation leads to inhibition of glycolysis, further reduction in ATP levels and cell death. Breast Cancer Res Treat. 2007 September; 105(1):17-28. Epub 2006 Nov. 17. PMID: 17111207; Cancer Biol Ther. 2008 Jan. 7; 7(4) [Epub ahead of print] PMID: 18305410.

The major characteristics of the trial outlined in Example 3 and the current, Phase IB trial (Example 5) are compared in the following table 5-1.

TABLE 5-1
BZL101 Phase 1A vs. BZL101 Phase 1B

| | Phase 1A (Ex. 3) | Phase 1B |
|---|---|---|
| Dose | Single Dose | Multiple Ascending Doses |
| | 12 g in 350 ml | 10 g in 100 ml; 20 g in 100 ml |
| | | 30 g in 150 ml; 40 g in 200 ml |
| | | (note: 20, 30, and 40 g were taken |
| | | twice/day) |
| # of | 21 | 27 |
| Participants | | |
| Study Drug | High volume | Reduced volume of insoluble plant fiber |
| | of insoluble | Taste - bitter taste has been modified |
| | plant fiber | and masked |
| | Taste - bitter | Freeze-dried to be mixed with liquid |
| | Liquid form | |

“g” = gram(s)

The major characteristics of the BZL101 Phase 1B cancer trial are summarized as follows:

BZL101 Phase 1B Design

Primary:

To determine the maximum tolerated dose of BZL101

To provide preliminary data on safety and efficacy of BZL101

Secondary:

Tumor response as defined by RECIST (Response Evaluation Criteria In Solid Tumors)

Overall and progression-free survival

Duration of response

Change in participant-reported QOL (EORTC QLQ-C30)

Main eligibility criteria:

Must have histologically confirmed breast cancer

Must have measurable stage IV disease

No more than 3 prior chemotherapies for metastatic disease (original unlimited #, amendment made mid-study for max of 3)

The escalating dose summary is presented in the following table 5-2:

TABLE 5-2
BZL101 Phase 1B Summary

| 10 grams dry | 20 grams dry | 30 grams dry | 40 grams dry |
|---|---|---|---|
| weight | weight | weight | weight |
| 10 g, q.d. | 10 g/b.i.d. | 15 g/b.i.d. | 20 g b.i.d. |
| 11 enrolled | 6 enrolled | 3 enrolled | 7 Enrolled |
| 1 DLT | 1 DLT | 0 DLT | 1 DLT |
| Average days on | Average days on | Average days on | Average days |
| study: 55 | study: 109 | study: 66 | on study: 28 |

“g” = gram(s);
“q.d.” = one administration per day;
“b.i.d.” = two administrations per day

The baseline characteristics for patients entering the study are as set forth in the following table 5-3:

TABLE 5-3
Phase 1B Baseline Characteristics

| Age (years) | N = 27 |
|---|---|
| Mean (SD) | 58.4 (13.9) |
| Median (Range) | 59 (32-78) |

Prior # of Cytotoxic Regimens for
Metastatic Disease

| | |
|---|---|
| Mean (SD) | 2.8 (2.4) |
| Median (Range) | 2 (0-10) |

Race/Ethnicity

| White/Caucasian | 16 (59%) |
|---|---|
| Black/African American | 6 (22%) |
| Latina/Hispanic | 5 (19%) |
| Hormone Receptor Status | N = 27 (%) |
| Positive (either ER or PR+) | 14 (63) |
| Negative (both ER and PR−) | 10 (37) |

HER2/neu Status

| | |
|---|---|
| Positive | 17 (63) |
| Negative | 10 (37) |

Baseline ECOG PS

| | |
|---|---|
| 0 | 16 (60) |
| 1 | 9 (33) |
| 2 | 2 (7) |

The demographic breakdown of the study participants is summarized in the following Table 5-4:

TABLE 5-4
Phase 1B Summary of Study Participants

| Study Participants Enrolled | N = 27 (%) |
|---|---|
| Included in safety analysis | 27 (100) |
| Evaluable by RECIST criteria | 18 (67) |
| Number of patients with DLTs | 3 (11) |
| Total number discontinued | 26 (96) |
| Disease progression | 18 (67) |
| Patient choice | 3 (19) |
| Adverse event | 2 (7) |
| Serious adverse event | 2 (7) |
| Non-compliance with study procedures | 1 (4) |

The number and type of adverse events experienced by the study participants are set forth in table 5-5, below:

TABLE 5-5
Phase 1B Adverse Events Related and Experienced by ≧10%

| Adverse | 10 g/d | 20 g/d | | | |
|---|---|---|---|---|---|
| Event By | N | N | 30 g/d N | 40 g/d N | Total N |
| CTCAE | (n = 11) | (n = 6) | (n = 3) | (n = 6) | (%) (n = 27) |
| Constitutional | 0 | 3 | 0 | 3 | 6 (22) |
| Fatigue | | | | | |
| Gastrointestinal | 1 | 2 | 0 | 0 | 3 (11) |
| Abdominal | | | | | |
| distension | | | | | |
| Diarrhea | 4 | 2 | 2 | 5 | 13 (48) |
| Flatulence | 1 | 1 | 0 | 1 | 3 (11) |
| Nausea | 2 | 2 | 2 | 5 | 11 (41) |
| Vomiting | 0 | 1 | 2 | 4 | 7 (26) |
| Metabolic/ | 2 | 1 | 1 | 0 | 4 (15) |
| Laboratory | | | | | |
| ALT elevation | | | | | |
| AST elevation | 2 | 1 | 0 | 0 | 3 (11) |
| Pain | 1 | 1 | 0 | 1 | 3 (11) |
| Pain-abdomen | | | | | |
| Headache | 3 | 1 | 0 | 0 | 4 (15) |

Phase 1B Dose Limiting Toxicities Definitions:
(a) Grade 3, 4, or 5 toxicity based on the NCI CTCAE V 3.0 that is possibly, probably, or definitely related to study medication
(b) Grade 2 gastrointestinal toxicity lasting for >3 weeks that is possibly, probably, or definitely related to study medication
(c) Baseline laboratory or medical conditions that worsen to grade 3 or above that is possibly, probably or definitely related to study medication

The dose limiting toxicities (DLTs) experienced by study participants are set forth in the following table 5-6:

TABLE 5-6
Phase 1B Dose Limiting Toxicities

| | # Days | | |
|---|---|---|---|
| ID # | on Study | Dose | Description |
| 03004 | 20 | 10 g/day | Grade 4 increase in AST. |
| 05003 | 19 | 20 g/day | Grade 3 diarrhea and fatigue deemed |
| | | | probably related. Note that this |
| | | | participant had a history of chronic |
| | | | diarrhea and was taking |
| | | | cholestryramine at baseline |
| | | | to treat this condition. |
| 05011 | 13 | 40 g/day | Grade 3 rib pain due to vomiting |
| | | | deemed definitely related. |
| | | | This participant had bone metastasis |
| | | | in her rib. |

Phase 1B Summary of Adverse Events:
a) BZL101 is well tolerated. The most common related adverse events are: diarrhea (48%), nausea (40%), vomiting (26%) and fatigue (22%).
b) There were 12 serious adverse events on the study, only 1 deemed related to study medication: hospitalization for grade 3 rib pain due to vomiting at the 40 g/day dose.
c) There were 3 patients with DLTs:
   (i) grade 4 AST elevation,
   (ii) grade 3 diarrhea and grade 3 fatigue in the same patient, and
   (iii) grade 3 rib pain due to vomiting.

Compliance with study medication is set forth in table 5-7, below:

TABLE 5-7
Phase 1B Compliance with Study Medication

| | 10 g/day | 20 g/day | 30 g/day | 40 g/day | Total |
|---|---|---|---|---|---|
| Compliance | N = 10* | N = 6 | N = 3 | N = 5* | N = 27 |
| Mean | 93% | 89% | 92% | 85% | 90% |
| Range | 73-113% | 61-101% | 85-100% | 79-96% | 61-113% |

*Note: compliance is unknown 1 participants at 10 g/day and 1 at 40 g/day

Phase 1B Preliminary Efficacy:
a) 21 of 27 were on trial for 28 days or more
b) 8/21 (38%) stable >90 days
c) 4/21 (19%) stable >180 days
d) 18 of 27 were are evaluable by RECIST (at least one measurable lesion and follow-up scan has been completed or is pending)
e) 6/18 (33%) stable >90 days
f) 3/18 (17%) stable >180 days

These results are summarized in the following table 5-8:

TABLE 5-8
Phase 1B Preliminary Efficacy

| | Hormone | | | |
|---|---|---|---|---|
| ID# | Receptor | # Days | # Days | |
| Dose | Status | on BZL | Stable | Comments |
| 03002 | ER− | 207 | 564 | Bone only disease (not evaluable by RECIST) At |
| 10 g/d | PR− | | | Month 2, the radiologist reported “Mild |
| | | | | improvement in the patient's bone scans with less |
| | | | | intrusive activity noted in the left anteromedial rib |
| | | | | and left acetabular region” |
| 05002 | ER+ | 124 | 418 | No scans repeated or cancer therapy started since |
| 10 g/d | PR− | | | stopping Jan 08. |
| 05005 | ER+ | 54 | 376 | Axillary tumor decreased from 2.5 cm at baseline to |
| 10 g/d | PR− | | | 1.5 cm at Month 1 on physical exam; breast tumor |
| | | | | also decreased in size at Month 1 on exam. Pending |
| | | | | independent radiology review to determine if |
| | | | | progressed. |
| 05006 | ER+ | 318 | 329 | Active on study. At Months 6 and 8 there was a 14% |
| 20 g/d | PR+ | | | and 16% decrease in total longest diameter, |
| | | | | respectively. At Month 10 there was a 16% |
| | | | | increase. |
| 05008 | ER− | 35 | 161 | Progressed based on clinical judgment, not by |
| 40 g/d | PR− | | | RECIST, so currently considered stable pending |
| | | | | Independent radiology review. At Month 1 there |
| | | | | was an 11.4% increase in total longest diameter |
| | | | | from baseline. |
| 03006 | ER+ | 130 | 137 | Despite progression noted in lung lesion at Month 4, |
| 20 g/d | PR− | | | bone scans demonstrated stable disease and patient |
| | | | | reported complete resolution of bone pain and |
| | | | | improved quality of life. |
| 07007 | ER+ | 113 | 113 | Progressed at Month 4. |
| 20 g/d | PR− | | | |
| 06003 | ER+ | 5 | 99 | Stopped BZL Aug. 2, 2008. Scans Sep. 25, 2008 indicated still |
| 40 g/d | PR+ | | | stable. |

“g” = gram(s);
“d” = day

Phase 2 Outcome Measures

Primary Outcomes

Obtain preliminary estimate of efficacy based on tumor response rate using RECIST Criteria

Adverse Events assessed at each clinic visit by self-report, physical exam and lab results

Secondary Outcomes
a. Tumor response: Clinical benefit rate, Complete response, Partial response, Progression of disease
b. Duration of response and survival time: Duration of overall response, complete response and partial response, Overall survival, and Progression-free survival
c. Change in quality of life using EORTC QLQ-C30

Summary

The MFD reached was 40 g/day. Phase 2 will move forward with 20 g/day enrolling 80 patients (40 HR+ and 40 HR−).

Extracts of Scutellaria Barbata inhibit the growth of breast cancer cells in vitro.

BZL101 treatment leads to the inhibition of glycolysis as evident from the decrease in the enzymatic activities within the glycolytic pathway and the inhibition of lactate production

BZL101 invokes selective cell death in cancer cells and not healthy cells

Oral administration of BZL101 is well tolerated. The most common adverse events are: diarrhea (48%), nausea (40%), vomiting (26%) and fatigue (22%)

There were 3 patients with DLTs: grade 4 AST elevation, grade 3 diarrhea and grade 3 fatigue in the same patient and grade 3 rib pain due to vomiting

One SAE was attributed to BZL101; hospitalization for the grade 3 rib pain due to vomiting at 40 g/day

On average, compliance with study medication was 90% of prescribed doses taken

In this heavily pre-treated population, 7/18 (39%) were stable for >90 days and 4/18 (22%) were stable for >180 days

Of the 27 women enrolled, 18 discontinued due to progression, 3 due to patient choice, 2 due to a an AE, 2 due to an SAE, and 1 due to non-compliance with study procedures.

From the foregoing, it can be seen that an extract of Scutellaria barbata D. Don, administered at a dose of 20 grams, 30 grams or 40 grams dry weight is effective and well tolerated for the treatment of metastatic breast cancer, and particularly metastatic breast cancer that has proven refractory to treatment.

From the foregoing, it is considered that daily doses of 15 grams dry weight to 60 grams dry weight of extract of Scutellaria barbata D. Don are effective in treating ER negative breast cancer, PR negative breast cancer, Her2/neu negative breast cancer and/or triple negative breast cancer, including those that have metastasized to other tissues. It is also considered that these doses are useful for the treatment of other ER negative, PR negative, Her2/neu negative and triple negative cancers. It is considered that doses of 20, 30 and 40 grams dry weight per day are particularly useful for treatment of the aforementioned cancers, especially ER negative breast cancer, PR negative breast cancer, Her2/neu negative breast cancer and/or triple negative breast cancer, including those breast cancers that have metastasized to other tissues.

Additional clinical trials of BZL101 can be carried out following the methodology set forth in Example 4. A patient who has been diagnosed with cancer is treated with 20 grams dry weight, 30 grams dry weight or 40 grams dry weight (or some other amount greater than 15 grams dry weight, e.g. from about 15-60 grams dry weight) of BZL101 and evaluated as set forth in Example 4, with appropriate modification depending upon the condition to be treated. Exemplary cancers to be treated include adrenal cortical cancer, anal cancer, aplastic anemia, bile duct cancer, bladder cancer, bone cancer, bone metastasis, Adult CNS brain tumors, Children CNS brain tumors, breast cancer, Castleman Disease, cervical cancer, Childhood Non-Hodgkin's lymphoma, colon and rectum (colorectal) cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyageal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine sacrcoma, vaginal cancer, vulvar cancer, Waldenstrom's macroglobulinemia, cancers of viral origin and virus-associated cancers.

### GENERAL CONCLUSION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising at least one excipient other than water, and one or more members of the group consisting of Luteolin, Apigenin, Scutellarein, and Scutellarin.

2. The pharmaceutical composition of claim 1, comprising each of Luteolin, Apigenin, Scutellarein, and Scutellarin, wherein at least one excipient other than water is selected from taste masking agents and sweeteners.

3. The pharmaceutical composition of claim 1, wherein the composition is substantially free of high molecular weight compounds.

4. The pharmaceutical composition of claim 1, wherein the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin.

5. The pharmaceutical composition of claim 1, wherein the composition contains about 1 g to about 200 g soluble matter, of which soluble matter about 1% to about 99% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin.

6. The pharmaceutical composition of claim 1, wherein the composition contains about 1 g to about 200 g soluble matter, of which soluble matter about 1.5% to about 2.1% is active soluble matter, of which active soluble matter about 1.7% to about 3.2% is Luteolin, about 2% to about 3.4% is Apigenin, about 7.9% to about 15.8% is Scutellarein, and about 49% to the balance of active soluble matter is Scutellarin.

7. A method of treating cancer, comprising administering to a cancer patient an effective amount of the composition of claim 1.

8. The method of claim 1, wherein said cancer is selected from breast cancer and one or more gynecological cancers.

9. The method of claim 8, wherein said cancer is a breast cancer.

10. The method of claim 9, wherein the breast cancer is advanced breast cancer, metastatic breast cancer, treatment-refractory breast cancer, ER-negative breast cancer, PR-negative breast cancer, HER2-negative breast cancer, and/or triple-negative breast cancer.

11. The method of one of claims 7, wherein the effective amount of the composition comprises at least 0.27 g of a combination of Luteolin, Apigenin, Scutellarein, and Scutellarin.

12. The method of claim 11, wherein the effective amount of the composition contains about 0.27 g to about 4 g of the combination of Luteolin, Apigenin, Scutellarein, and Scutellarin.

13. The method of claim 12, wherein the effective amount of the composition contains about 0.35 g to about 4 g of the combination of Luteolin, Apigenin, Scutellarein, and Scutellarin.

14. The method of claim 11, wherein the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin.

15. A dosage unit comprising at least about 0.25 g of a combination of Luteolin, Apigenin, Scutellarein, and Scutellarin.

16. The dosage unit of claim 15, comprising at least about 0.35 g of a combination of Luteolin, Apigenin, Scutellarein, and Scutellarin.

17. The dosage unit of claim 15, comprising about 0.35 g to about 4 g of the combination of Luteolin, Apigenin, Scutellarein, and Scutellarin.

18. The dosage unit of one of claim 15, wherein the dosage unit further comprises at least one excipient other than water.

19. The dosage unit of claim 18, wherein at least one excipient other than water is a taste masking agent, a sweetener or both.

20. The dosage unit of claim 15, wherein the dosage unit is substantially free of high molecular weight compounds extracted from Scutellaria barbata D. Don.

21. The dosage unit of claim 15, wherein the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin.

22. A method of treating cancer, comprising administering to a cancer patient at least about 0.25 g per day of a combination of Luteolin, Apigenin, Scutellarein, and Scutellarin.

23. The method of claim 22, wherein said cancer is selected from breast cancer and one or more gynecological cancers.

24. The method of claim 23, wherein said cancer is a breast cancer.

25. The method of claim 24, wherein said breast cancer is advanced breast cancer, metastatic breast cancer, treatment-refractory breast cancer, ER-negative breast cancer, PR-negative breast cancer, HER2-negative breast cancer, and/or triple-negative breast cancer.

26. The method of claim 22, comprising administering to the patient at least about 0.35 g per day of a combination of Luteolin, Apigenin, Scutellarein, and Scutellarin.

27. The method of claim 26, comprising administering to the patient about 0.35 g to about 4 g per day of a combination of Luteolin, Apigenin, Scutellarein, and Scutellarin.

28. The method of claim 22, wherein the combination of Luteolin, Apigenin, Scutellarein, and Scutellarin contains about 1 part Luteolin, about 0.61 to about 2 parts Apigenin, about 2.5 to about 9.4 parts Scutellarein, and about 15 to about 70 parts Scutellarin.

29. A pharmaceutical composition comprising 1 part of a combination of Luteolin, Apigenin, Scutellarein, and Scutellarin and less than about 50 parts of high molecular weight compounds having molecular weights greater than a predetermined cutoff, wherein the predetermined cutoff is from 1,000 gs/mole to about 20,000 gs/mole.

30. The pharmaceutical composition of claim 29, comprising 1 part of the combination of Luteolin, Apigenin, Scutellarein, and Scutellarin and less than about 40 parts of the high molecular weight compounds.

31. The pharmaceutical composition of claim 29, wherein the cutoff for the high molecular weight compounds is from 1,000 to 10,000 gs/mole.

32. The pharmaceutical composition of claim 29, further comprising at least one excipient other than water.

33. The pharmaceutical composition of claim 32, wherein at least one excipient other than water is a taste masking agent, a sweetener or both.

34. A dosage unit comprising a pharmaceutical composition of one of claim 29 that contains at least about 18 μg of a combination of Luteolin, Apigenin, Scutellarein, and Scutellarin.

35. The dosage unit of claim 34, containing about 0.25 g to about 4 g of the combination of Luteolin, Apigenin, Scutellarein, and Scutellarin.

36. The dosage unit of claim 34, containing about 0.27 g to about 4 g of the combination of Luteolin, Apigenin, Scutellarein, and Scutellarin.

37. The dosage unit of claim 34, containing about 0.35 g to about 4 g of the combination of Luteolin, Apigenin, Scutellarein, and Scutellarin.

38. The dosage unit of claim 34, further comprising at least one excipient other than water.

39. The dosage unit of claim 38, wherein at least one excipient other than water is selected from taste masking agents, sweeteners, or both.

40. A method of treating cancer comprising administering to a cancer patient an effective amount of a pharmaceutical composition of claim 29.

41. The method of claim 40, wherein the cancer is one or more breast cancers and/or gynecological cancers.

42. The method of claim 41, wherein the cancer is a breast cancer.

43. The method of claim 42, wherein the breast cancer is advanced breast cancer, metastatic breast cancer, treatment-refractory breast cancer, ER-negative breast cancer, PR-negative breast cancer, HER2-negative breast cancer, and/or triple-negative breast cancer.

44. A method of treating cancer comprising administering to a cancer patient an effective amount of a dosage unit of claim 34.

45. The method of claim 44, wherein the cancer is one or more breast cancers and/or gynecological cancers.

46. The method of claim 45, wherein the cancer is a breast cancer.

47. The method of claim 46, wherein the breast cancer is one or more of advanced breast cancer, metastatic breast cancer, treatment-refractory breast cancer, ER-negative breast cancer, PR-negative breast cancer, HER2-negative breast cancer, and/or triple-negative breast cancer.

48. A process of making a pharmaceutical composition, comprising:
(a) contacting aerial parts of Scutellaria barbata D. Don with water heated to above 40° C. for a period at least about 10 minutes to form a mixture;
(b) separating the aerial parts of Scutellaria barbata D. Don from the mixture to produce a crude extract;
(c) separating high molecular weight compounds from the crude extract to form a refined extract;
(d) optionally evaporating some, substantially all or all of the water from the refined extract or adding additional water to the refined extract; and
(e) combining the refined extract with at least one pharmaceutically acceptable excipient other than water, to form the pharmaceutical composition.

49. A process of making a pharmaceutical composition, comprising:
(a) contacting aerial parts of Scutellaria barbata D. Don with water heated to above 40° C. for a period at least about 10 minutes to form a mixture;
(b) separating the aerial parts of Scutellaria barbata D. Don from the mixture to produce a crude extract;
(c) separating high molecular weight compounds from the crude extract to form a refined extract;
(d) optionally evaporating some, substantially all or all of the water from the refined extract or adding additional water to the refined extract; and
(e) combining the refined extract with a pharmaceutically acceptable excipient to form the pharmaceutical composition.

50. A process of making a refined extract of Scutellaria barbata D. Don, comprising:
(a) contacting aerial parts of Scutellaria barbata D. Don with water heated to above 40° C. for a period at least about 10 minutes to form a mixture;
(b) separating the aerial parts of Scutellaria barbata D. Don from the mixture to produce a crude extract; and
(c) separating high molecular weight compounds from the crude extract to form the refined extract of Scutellaria barbata D. Don.

51. A process of making a pharmaceutical composition, comprising combining at least one pharmaceutically acceptable excipient other than water with one or more members of the group consisting of Luteolin, Apigenin, Scutellarein, and Scutellarin to form the pharmaceutical composition.

52. A process of making a pharmaceutical dosage unit comprising:
(a) contacting aerial parts of Scutellaria barbata D. Don with water heated to above 40° C. for a period at least about 10 minutes to form a mixture;
(b) separating the aerial parts of Scutellaria barbata D. Don from the mixture to produce a crude extract; and
(c) separating high molecular weight compounds from the crude extract to form a refined extract; and
(d) combining the refined extract with at least one excipient other than water to form the pharmaceutical dosage unit.
